(19) United States
(12) Patent Application Publication
(10) Pub. No.: US 2024/0081996 A1
(43) Pub. Date: Mar. 14, 2024

(54) ANNULOPLASTY DEVICE, CINCHING DEVICE AND METHOD OF ANNULOPLASTY

(71) Applicant: Silara Medtech Inc., Santa Rosa, CA (US)

(72) Inventors: Do D. UONG, Santa Rosa, CA (US); Trevor M. GREENAN, Santa Rosa, CA (US); Mathew A. HAGGARD, Santa Rosa, CA (US); Luis A. HERNANDEZ, Santa Rosa, CA (US); Leonardo RODRIGUEZ, Santa Rosa, CA (US)

(21) Appl. No.: 18/303,438

(22) Filed: Apr. 19, 2023

Related U.S. Application Data
(63) Continuation of application No. PCT/US2021/054688, filed on Oct. 13, 2021.
(60) Provisional application No. 63/093,637, filed on Oct. 19, 2020.

(51) Int. Cl. A61F 2/24

(52) U.S. Cl. CPC A61F 2/2445; A61F 2/2466; A61F 2230/0006; A61F 2250/001; A61F 2250/0026; A61F 2250/006

(57) ABSTRACT

The present disclosure relates to an annuloplasty device configured for being implanted via a catheter, a cinching device applied in annuloplasty, and a method of implementing annuloplasty. The annuloplasty device comprises a shaping ring and a tissue anchoring element. The shaping ring includes a first bar member, a second bar member and a third bar member that are connected via an extendable and compressible element. The first bar member, the second bar member and the third bar member are each provided with a connection portion for anchoring the shaping ring to the physiological tissue by the tissue anchoring element. An adjustment wire is provided in an inner cavity of the extendable and compressible element, for adjusting the size of the shaping ring, thereby adjusting the size of the physiological annulus.

### CROSS-REFERENCE TO RELATED APPLICATIONS

This application is a continuation of International Application No. PCT/US2021/054688, filed on Oct. 13, 2021, which claims the benefit of priority to U.S. Provisional Application No. 63/093,637, filed on Oct. 19, 2020, each of which are herein incorporated by reference in its entirety for all purposes.

### FIELD

The present disclosure relates to the field of annuloplasty, and in particular, to a transcatheter-implanted annuloplasty device, a cinching device for annuloplasty, and a method of implementing the annuloplasty.

### BACKGROUND

The mitral valve is located at the junction between the left atrium and the left ventricle of the heart. During diastole, the mitral valve opens to allow blood to flow from the left atrium to the left ventricle. During systole, when the left ventricle pumps blood through the aorta into the body, the mitral valve closes to prevent backflow of the blood into the left atrium. The mitral valve consists of two leaflets (posterior and anterior leaflets) located on the mitral annulus. The mitral annulus is an annulus that forms the connection between the left atrium and the left ventricle. The leaflets of the mitral valve are tethered to the papillary muscles of the left ventricle via chordae tendineae. Chordae tendineae can prevent the mitral leaflets from averting into the left atrium during systole.

Mitral regurgitation is caused by the failure of both leaflets of the mitral valve to close completely, with the result that blood flows back from the left ventricle to the left atrium during systole. Regurgitation may also be due to dilation of the mitral annulus. For example, regurgitation is caused by an increase in the anteroposterior diameter of the mitral annulus. In addition, left ventricular dilatation also causes mitral regurgitation. For example, the occurrence of an infarction may cause the left ventricle to dilate and cause mitral regurgitation. In addition, left ventricular dilatation further causes the papillary muscles to continuously bind the mitral leaflet into an open configuration via the chordae tendineae, resulting in regurgitation of the mitral valve due to mitral insufficiency.

The tricuspid valve is located at the junction between the right atrium and the right ventricle of the heart. During diastole, the tricuspid valve opens, allowing blood to flow from the right atrium into the right ventricle. During systole, when the right ventricle pumps blood through the pulmonary artery into the lungs, the tricuspid valve closes to prevent backflow of blood into the right atrium. Tricuspid valve is composed of an anterior leaflet, a posterior leaflet and a septal leaflet. The tricuspid valve hangs toward the chamber of the right ventricle and is connected to the papillary muscles in the right ventricular wall by chordae tendinae. Similar to the mitral valve, the tricuspid valve may also have regurgitation due to insufficiency.

There are currently many devices and methods for treating mitral regurgitation. These devices and methods primarily include replacement or repair of the mitral valve. Replacement of the mitral valve is usually performed through transapical or transseptal procedures. There are usually four types of mitral valve repair: valve leaflet clip; direct annuloplasty; indirect annuloplasty; and chordae tendineae repair. Both direct and indirect annuloplasty involve reshaping the mitral annulus and/or left ventricle of a subject so that the anterior and posterior leaflets are properly closed to prevent regurgitation by eliminating mitral insufficiency. For some annuloplasty applications, a shaped ring is implanted in the vicinity of the mitral annulus, the purpose of which is to reduce the circumference of the mitral annulus so that the anterior and posterior leaflets are brought closer to prevent regurgitation. The tricuspid valve can also be replaced or repaired by similar devices and methods.

### SUMMARY OF THE DISCLOSURE

An object of the present disclosure is to provide an improved technology for transcatheter annuloplasty that makes the operation of annuloplasty simpler and more reliable.

According to a first aspect of the present disclosure, there is provided an annuloplasty device configured for being implanted via a catheter, comprising: a shaping ring including an extendable and compressible element and at least one bar member connected to the extendable and compressible element, the at least one bar member is provided with at least one connection portion; and a tissue anchoring element configured to fix the at least one bar member to an annulus tissue via the connection portion.

In this embodiment, since the bar members of the shaping ring are connected by the extendable and compressible element, the entire shaping ring can be delivered into the body through the catheter. In addition, due to the expansion and contraction characteristics of the extendable and compressible element, the shaping ring may automatically expand into its original shape within the body via the extendable and compressible element after deployment of the shaping ring from the catheter. Due to the arrangement of the bar member, the shaping ring can be brought to a target position by coupling a torque drive device inside the steerable sheath with the bar member after the shaping ring is deployed into its original shape. In addition, due to the arrangement of the bar member, it is possible to control the position of the bar member by single manipulation of the sheath, thereby completing the delivery of a plurality of tissue anchoring elements corresponding to each bar member. Therefore, the operation of the shaping ring of this embodiment is simple, and the time required for the operation in the heart during the implantation procedure can be reduced.

According to a second aspect of the present disclosure, there is provided an annuloplasty device configured for being implanted via a catheter, comprising: a shaping ring including an extendable and compressible element and at least one bar member connected to the extendable and compressible element, the at least one bar member is provided with at least one connection portion; and a tissue anchoring element configured to fix the at least one bar member to the annulus tissue via the connection portion, wherein the connection portion includes an abutment fixed to a hole wall of a through hole of the bar member and a cylindrical anchor alignment member aligned with the through hole, the cylindrical anchor alignment member is configured to cause the tissue anchoring element to be substantially perpendicular to a bottom surface of the at least one bar member in contact with the annulus tissue when the tissue anchoring element is anchored.

According to a third aspect of the present disclosure, there is provided a method of implementing annuloplasty, the method comprising: connecting a guide to a guide-engaging element of a shaping ring; folding the entire shaping ring into the catheter; introducing the shaping ring and surgical apparatuses through the catheter into the body; anchoring the bar member of the shaping ring to the annulus tissue by the tissue anchoring element; contracting the annulus to a proper size by adjusting an adjustment wire; locking the adjustment wire to keep the annulus properly sized; and withdrawing the catheter and the surgical apparatuses.

In the method, after the entire shaping ring is delivered through the catheter into the heart (e.g., the left atrium), upon adjustment and control of the position and orientation of the bar member, the positioning, anchoring and adjustment of the shaping ring can be accomplished efficiently. The method of implementing annuloplasty provided by the present disclosure is simple, and the time required for the operation in the heart during the implantation procedure can be reduced.

The present disclosure will be described in more detail below with the accompanying drawings.

### BRIEF DESCRIPTION OF THE DRAWINGS

The features and advantages of the above aspects of the disclosure will be more clearly understood from the following detailed description of exemplary embodiments given in conjunction with the accompanying drawings, which illustrate by way of non-limiting example, exemplary embodiments of the disclosure, among which:

FIG. 1A is a structural schematic view showing a shaping ring for annuloplasty according to an embodiment of the present disclosure, in which the connection among an extendable and compressible element, an adjustment wire and a bar member is shown in an enlarged view;

FIG. 1B is an example showing a shaping ring including a single extendable and compressible element;

FIGS. 2A and 2B are perspective views illustrating examples of the bar member according to the present disclosure;

FIGS. 3A and 3B are partial front views showing alternative examples of a ring of the bar member;

FIG. 4 is a perspective view of a tissue anchoring element in accordance with the present disclosure;

FIG. 5 is a perspective view of an example of a guide-engaging element according to the present disclosure;

FIG. 6 is a perspective view of an example of a guide according to the present disclosure;

FIG. 7 shows a schematic diagram of the fitting between a torque drive device and a projection for use during implantation of a shaping ring according to the present disclosure;

FIG. 8A is perspective view illustrating an example of another bar member according to the present disclosure;

FIG. 8B is a perspective view illustrating a base of the bar member shown in FIG. 8A;

FIG. 8C is a perspective view showing an example of a deployed state of a foldable bar member according to the present disclosure;

FIG. 8D is a perspective view showing an example of a folded state of the bar member shown in FIG. 8C;

FIGS. 9A and 9B are perspective views of a housing of a cinching device disposed on a bar member of a shaping ring of the present disclosure;

FIGS. 10A, 10B and 10C are perspective views and a cross-sectional view of a reel of a cinching device disposed on a bar member of a shaping ring of the present disclosure, respectively;

FIGS. 11A, 11B, 11C and 11D are a perspective view, a front view, a top view and a side view of the elastic sheet of the cinching device, respectively;

FIG. 12 is a perspective view showing the fitting between the torque drive device and the cinching device;

FIGS. 13A and 13B are sectional perspective views showing the cinching device in a one-way locked state and in an unlocked state, respectively;

FIG. 13C is a cross-sectional perspective view showing that a torque drive device drives the cinching device to rotate while in an unlocked state;

FIGS. 14A and 14B are schematic views respectively illustrating shaping rings with fabrics and with a portion of the fabrics removed in a natural expanded state according to one embodiment of the present disclosure;

FIGS. 14C and 4D are schematic views respectively illustrating shaping rings with fabrics and with a portion of the fabrics removed in a contracted state according to one embodiment of the present disclosure;

FIG. 15A is a structural diagram showing a shaping ring without fabrics in a natural expanded state in accordance with another embodiment of the present disclosure, in which a segmented extendable and compressible element and a continuous adjustment wire are employed;

FIG. 15B is a structural diagram showing a shaping ring without fabrics in a natural expanded state in accordance with a further embodiment of the present disclosure, in which a segmented extendable and compressible element and a segmented adjustment wire are employed;

FIG. 16A is a schematic diagram of the shaping ring with fabrics in a natural expanded state as shown in FIG. 15A;

FIG. 16B is a schematic diagram of an annuloplasty device with fabrics and a helical tissue anchoring element as shown in FIG. 1A or 1B;

FIG. 16C is a schematic diagram of the shaping ring with fabrics in a contracted state as shown in FIG. 15A;

FIGS. 17A, 17B, 17C are schematic views respectively showing the shaping ring fully folded in a catheter, partially folded in a catheter, and released from the catheter to be in a natural expanded state according to one embodiment of the present disclosure;

FIG. 18 is a general schematic diagram showing the structure of a human mitral valve;

FIGS. 19A to 19D are schematic diagrams illustrating adjusting the position or orientation of a shaping ring relative to a physiological annulus in accordance with the present disclosure;

FIGS. 20A to 20F are schematic diagrams illustrating the effect of adjusting a mitral valve annulus by an annuloplasty device according to one embodiment of the present disclosure;

FIGS. 21A to 21F are schematic diagrams illustrating the effect of adjusting a mitral valve annulus by an annuloplasty device according to another embodiment of the present disclosure;

FIG. 22 is a diagram illustrating one exemplary process of performing an annuloplasty using an annuloplasty device according to one embodiment of the present disclosure;

FIGS. 23 to 35 specifically illustrate the process of implanting an annuloplasty device according to one embodiment of the present disclosure corresponding to FIG. 1A or FIG. 1, viewed in a generally caudal direction through a left atrium at a mitral valve, and illustrate the steps of the exemplary method outlined in FIG. 22; and

FIGS. 36 and 37 specifically illustrate the process of implanting an annuloplasty device according to another embodiment of the present disclosure as shown in FIG. 15A, viewed in a generally caudal direction through a left atrium at a mitral valve, and illustrate different steps with respect to the steps of implanting the shaping ring corresponding to FIG. 1A or 1B.

### DETAILED DESCRIPTION

Exemplary embodiments of the present disclosure based on mitral valve repair are described in detail below with reference to the accompanying drawings, for which repair of the tricuspid valve may be performed in a manner similar to annuloplasty of the mitral valve. It should be understood that embodiments having other arrangements may be employed without departing from the scope of the present disclosure. Accordingly, the following detailed description should not be construed as limiting the scope of the present disclosure. The scope of the present disclosure is defined by the appended claims and their equivalents. In all the drawings, the same reference numerals denote elements having the same or similar functions.

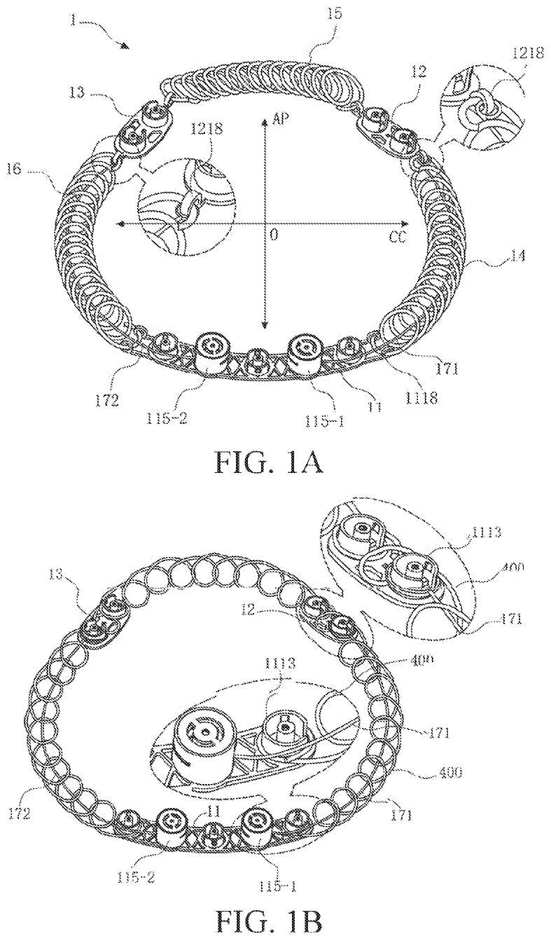

FIG. 1A shows a shaping ring 1 for annuloplasty according to one embodiment of the present disclosure. For convenience of explanation, the direction of the axis passing through the center O of the shaping ring 1 in an anterior-posterior direction is defined as the AP axis direction, and the direction passing through the center O and perpendicular to the AP axis direction is defined as the CC axis direction.

As shown in FIG. 1A, the shaping ring 1 is a complete ring comprising bar members 11, 12, 13, extendable and compressible elements 14, 16 and an optional interconnecting element 15. The bar members 11, 12, 13 can be connected to one another via respective extendable and compressible elements 14, 16. The bar members 12, 13 may also be further connected by an interconnecting element 15 so that the shaping ring 1 forms a complete substantially annular shape. In the example shown in FIG. 1A, the extendable and compressible elements 14, 16 are connected to the respective bar members 11, 12, 13 by the respective free ends being hooked to the respective loops 1118, 1218 of the bar members 11, 12, 13. The interconnecting element 15 may also interconnect the bar members 12, 13 by hooking its respective free ends onto the respective loops 1218 of the bar members 12, 13. Alternatively or additionally, the free ends of the extendable and compressible elements or the optional interconnecting element may be further welded to the respective loops 1118, 1218.

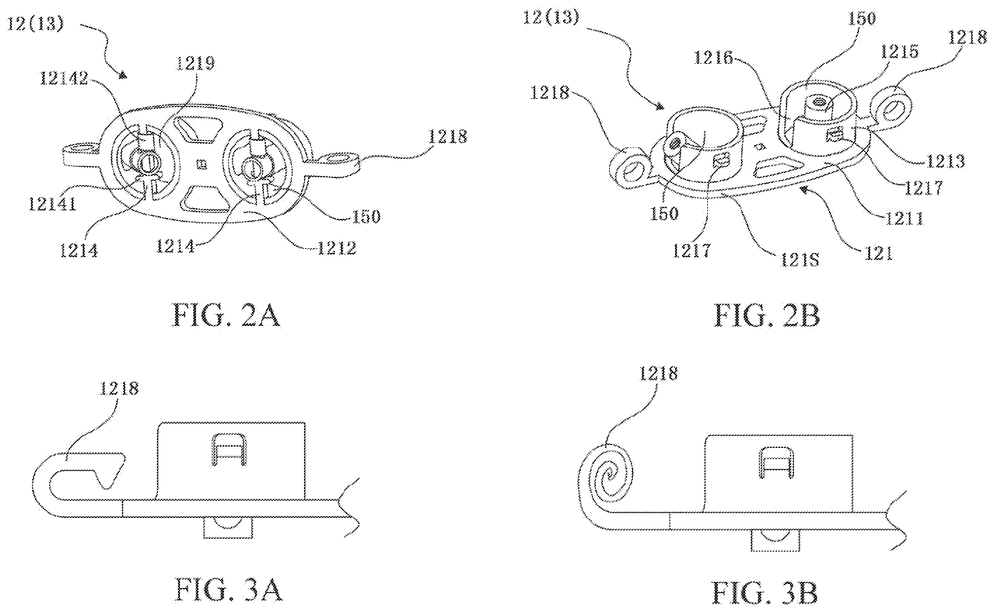

As shown in FIGS. 1A, 2A, 2B and 8A, two loops 1118 are provided at each end of the bar member 11, and one loop 1218 is provided at each end of the bar members 12, 13. The bar members 11 to 13 may also be provided with other numbers of loops. In addition, as shown in FIGS. 2A and 2B, the loop 1218 may be a closed loop. Alternatively, the loop 1218 may be a partially closed loop, as shown in FIGS. 3A and 3B. The loop 1118 of the bar member 11 may take the same or different form as the loop 1218.

Continuing with reference to FIG. 1A, the shaping ring 1 further comprises two adjustment wires 171, 172. One end of the adjustment wire 171 is connected to the loop 1218 of the bar member 12, and the other end thereof is connected to the cinching device 115-1 provided on the bar member 11. One end of the adjustment wire 172 is connected to the loop 1218 of the bar member 13, and the other end thereof is connected to the cinching device 115-2 provided on the bar member 11. When the respective adjustment wires 171, 172 are tightened by the cinching devices 115-1, 115-2, the adjustment wire 171 pulls the bar members 11, 12 toward each other, and the adjustment wire 172 pulls the bar members 11, 13 toward each other.

In the example shown in FIG. 1A, the adjustment wire 171 extends within the interior cavity defined by the extendable and compressible element 14, and the adjustment wire 172 extends within the interior cavity defined by the extendable and compressible element 16. Alternatively, the adjustment wires 171, 172 may extend at least partially within the interior cavity defined by the respective extendable and compressible elements. For example, in the case where the extendable and compressible element is a coil element, the adjustment wires 171, 172 may alternately pass through the coils of the coil element. The advantage of this arrangement is that the coil element can better snugly abut against the annulus tissue. Alternatively, the adjustment wires 171, 172 may also extend outside the extendable and compressible element.

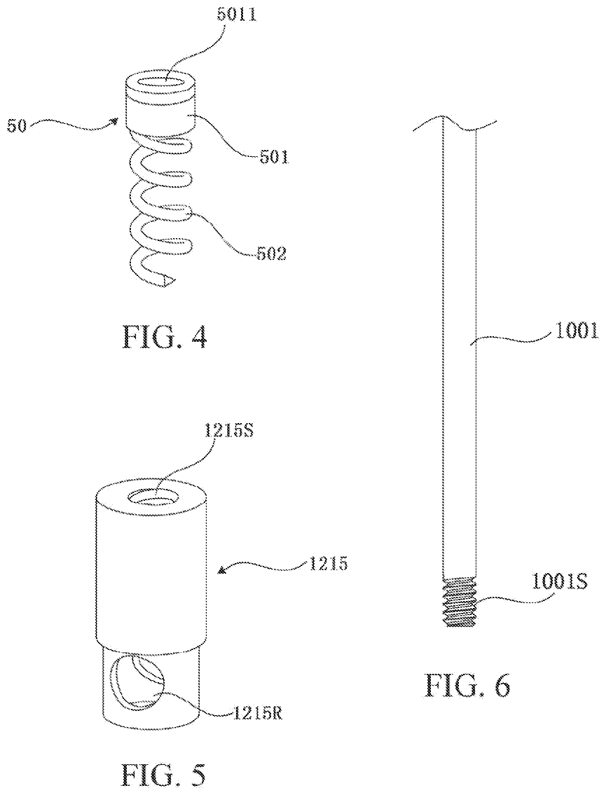

No adjustment wire is provided inside or outside the interior cavity defined by the interconnecting element 15. The interconnecting element 15 may comprise a linear element, such as a wire, composed of a shape memory material. Preferably, the interconnecting element 15 is constituted by an inclined flat-shaped coil element, the coils of which can be configured to be inclined with respect to the longitudinal centre line of the interconnecting element 15, thereby enabling the interconnecting element 15 to snugly abut against the surface of the annulus tissue. It is further preferred that when the bar members 2, 3 are connected by means of an interconnecting element 15, the interconnecting element 15 snugly conforms to the physiological annulus at the anterior leaflet of the annulus tissue, i.e. the shape of the interconnecting element 15 can be adapted to the physiological structure at the anterior leaflet. For example, the interconnecting element 15 may have a saddle shape suitable for repairing a mitral valve. A tissue anchoring element (e.g., the tissue anchoring element shown in FIG. 4) may be further provided at the interconnecting element 15 to cause the interconnecting element 15 to further snugly conforms to the physiological annulus at the anterior leaflet of the mitral valve.

As an alternative arrangement shown in FIG. 1A, there may be no connection between the bar members 12, 13, i.e. no interconnecting element 15 is provided, so that the shaping ring 1 is formed as a non-complete ring. If the bar members 12, 13 are connected, it is possible to reduce the requirements for positioning accuracy during the operation, to simplify the operation, and to reduce the operation time in vivo. Thus, in the preferred example shown in FIG. 1A, the bar members 12, 13 are connected by an interconnecting element 15. The annuloplasty device constituted by the shaping ring 1 being fixed to the tissue by the tissue anchoring element may provide a docking structure for receiving a prosthetic valve for subsequent corresponding valve replacement.

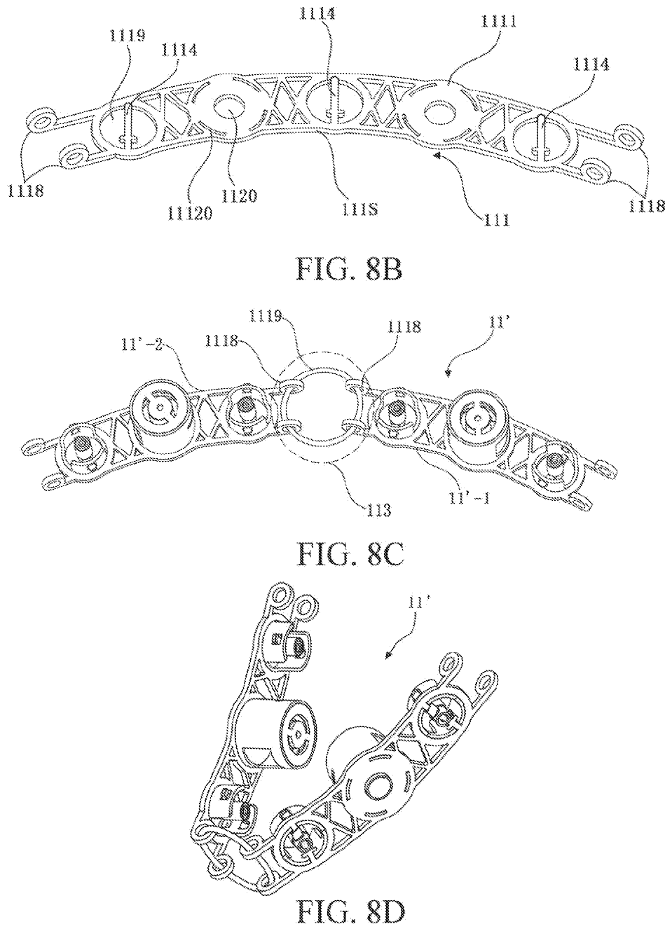

Referring to FIGS. 2A and 8A, the bar members 11, 12 and 13 are each provided with at least one connection portion 150 configured to be connected directly to a tissue anchoring element when the tissue anchoring element is anchored to the annulus tissue, thereby securing the respective bar members to the annulus tissue. In the example of FIG. 1A, the bar member 11 is provided with three connection portions 150 (FIG. 8A), and the bar members 12 and 13 are provided with two connection portions 150 (FIGS. 2A and 2B). Of course, the bar members 11, 12, 13 may be provided with a different number of connection portions according to actual needs.

As shown in FIGS. 2A and 8B, the connection portion 150 may include a cross bar 1214 (FIG. 2A) and a cross bar 1114 (FIG. 8B) that are radially fixed to the hole walls of the through holes 1219, 1119 provided in the respective bar members. The cross bars 1114, 1214 may be radially fixed by welding to the hole walls of the through holes 1119, 1219 provided in the respective bases 111, 121 of the bar members 11, 12 or 13.

Continuing with reference to FIG. 1A, the bar member 11 may include cinching devices 115-1, 115-2, each of which may be configured to adjust (tighten or release) the adjustment wires 171, 172, thereby adjusting the size of the annulus of the shaping ring 1, which in turn regulates the size of the physiological annulus. In the example shown in FIG. 1A, both cinching devices 115-1, 115-2 are provided on the bar member 11. Alternatively, the cinching devices 115-1, 115-2 may be provided on the bar members 12, 13, respectively, or one on the bar member 11 and the other on the bar member 12 or 13. The advantages of providing the cinching device on the bar member at least include that the cinching device can be installed to the corresponding bar member in vitro in advance and the corresponding adjustment wire can be connected in advance, thereby reducing the time for in-vivo surgery. For example, one end of the adjustment wire 171 may be fixed to one end of the bar member 12 (for example, the loop 1218) in vitro, and the other end of the adjustment wire 171 may be fixed to the cinching device 115-1 on the bar member 11 (for example, through the adjustment wire hole 11528 of the reel 1152 of the cinching device and fixed to the cinching device 115-1, for example by means of knotting). The adjustment wire 172 may be provided in a similar manner.

It should be noted that the cinching devices 115-1, 115-2 and other cinching devices mentioned below operate in the same manner, and only the cinching device 115-1 is taken as an example for description.

The reel 1152 (see FIGS. 10A to 10C) of the cinching device 115-1 may be driven to rotate in the tightening direction and the release direction. When the reel 1152 of the cinching device 115-1 is driven to rotate in the tightening direction, the adjustment wire 171 is wound around the reel 1152 with the rotation of the reel 1152, thereby shortening the length of the adjustment wire 171, thereby pulling the bar members 11 and 12 toward each other. Similarly, when the reel 1152 of the cinching device 115-2 is rotated, the length of the adjustment wire 172 is also shortened, thereby pulling the bar members 11 and 13 toward each other. Thus, the annular surface of the annular shaping ring 1 is reduced. Because the bar members 11, 12, 13 are connected to the physiological valve annulus by means of tissue anchors or anchoring elements at the location of their respective connection portions, the physiological valve annulus shrinks as the annulus of the shaping ring 1 shrinks, thereby reducing the circumference of the physiological valve annulus and bringing the anterior and posterior leaflets closer to each other. In this way, the regurgitation caused by insufficient closure of the anterior and posterior leaflets can be eliminated.

In the annuloplasty device of the present disclosure, because no adjustment wire is provided at the interconnecting element 15 between the bar members 12 and 13, or no interconnecting element 15 may even be provided between the bar members 12 and 13, it is possible to avoid the crinkling of the anterior leaflet of the mitral valve which affects the opening and closing function of the normal physiological leaflet, and to avoid affecting the normal function of the aortic valve behind the mitral curtain.

In the present embodiment, coil elements are employed as the extendable and compressible elements 14, 16 and the interconnecting element 15. In addition to defining the interior cavity for arranging the adjustment wire, the coil of the coil element may also be inclined with respect to the longitudinal central axis of its interior cavity such that the entire coil element is flattened, thereby enabling the extendable and compressible element or the interconnecting element to snugly abut against the surface of the valve annulus tissue. For example, the coil of the coil element may be at an angle of 0° to 45°, preferably 0° to 30°, more preferably 0° to 15°, or even less relative to the longitudinal central axis. In this way, in addition to its stretchable and contractible properties, the extendable and compressible element can also fit closely to the physiological annulus, thereby being able to maintain normal three-dimensional motion and enable the mitral annulus to function optimally. In the example shown in FIG. 1A, no adjustment wire is provided in the interior cavity of the interconnecting element 15, and therefore, the interconnecting element 15 remains substantially in a natural extended state before and after adjustment.

In the example shown in FIG. 1A, because the annuloplasty device is provided with bar members 11, 12, 13 connecting the shaping ring 1 to the annulus tissue, and each bar member is provided with a connection portion 150 connected to the annulus tissue by a tissue anchoring element at a anchoring position, the extendable and compressible elements 14, 16 and optionally the interconnecting element 15 may be connected to the respective bar members in vitro prior to implantation of the shaping ring 1, and the cinching devices 115-1, 115-2 are rotatably fixed to the respective bar members (in the example of FIG. 1A, to the bar member 11) in vitro. Furthermore, the adjustment wires 171, 172 may also be connected to the respective bar members and the cinching devices in vitro. In this way, after implantation of the shaping ring 1, it is only necessary to connect the bar members to the annulus tissue by means of the tissue anchoring elements through the connection portion provided on the bar members. Thus, in addition to the operation of tensioning the shaping ring, the surgical operation required in vivo only involves anchoring the tissue anchoring elements and connecting the entire shaping ring to the annulus tissue via the connection portions of the bar members by the tissue anchoring elements. Thus, the procedure of the surgical operation is simple, reliable and requires less time.

Referring to FIGS. 2A and 2B, an example of the bar members 12, 13 is shown. The bar members 12, 13 may be identical strip-shaped elements or elements of other shapes, or may be different strip-shaped elements or elements of other different shapes. For example, the bar members 12, 13 may be provided with a different number of connection portions 150 and cinching devices 115-1, 115-2 and/or different shapes.

As shown in FIGS. 2A and 2B, the bar members 12 and/or 13 may include a flat plate-shaped base 121, which may have an oblong shape. However, the base 121 may have a shape other than an oblong shape, such as a circular shape, an oval shape, or a polygonal shape or the like. The base 121 has a first surface 1211 and a second surface 1212 substantially parallel and opposite to the first surface 1211. The circumferential side 121S extends around the periphery of the base 121 between the first surface 1211 and the second surface 1212, thereby connecting the first surface 1211 and the second surface 1212. When the shaping ring 1 is anchored to the physiological valve annulus, the second surface 1212 opposes and contacts the physiological valve annulus tissue, thereby providing a relatively large anchoring surface for the shaping ring 1.

The base 121 may be provided with at least one connection portion 150. FIG. 2B shows that the base 121 is provided with two connection portions 150. Of course, the base 121 may be provided with other numbers of connection portions 150 as needed.

As shown in FIGS. 2A and 2B, a cylindrical anchor alignment member 1213 extending perpendicularly from the first surface 1211 is fixedly provided at the location of each connection portion 150, the cylindrical wall of the cylindrical anchor alignment member defines an inner cylindrical cavity, the inner cylindrical cavity extends through the base 121 to the second surface 1212 so as to open into the second surface 1212. By providing an anchor alignment member substantially perpendicular to the first surface 1211 of the base 121, it is possible to ensure that the tissue anchoring element (e.g., the helical tissue anchoring element shown in FIG. 4) is substantially perpendicular to the second surface 1212 of the base 121 throughout the anchoring process. Thus, the anchoring element can be aligned with the tissue without having to adjust a specific angle by manipulating the catheter, thereby simplifying the procedure and making the anchoring more reliable and less time consuming.

In this example, the anchor alignment member 1213 is formed integrally with the base 121. However, the anchor alignment member may also be separately formed and fixed to the base 121 by a process such as welding. In this case, it is necessary to provide a corresponding through hole 1219 in the base in advance, the anchor alignment member 1213 may be received in the through hole or may be fixed to the first surface 1211 of the base 121 in alignment with the edge of the through hole.

In the example shown in FIGS. 2A and 2B, a cross bar 1214 is provided in the interior cavity of the cylindrical anchor alignment member 1213, and the cross bar 1214 is radially fixed to the cylindrical wall of the cylindrical anchoring member. In FIG. 2B, the cross bar 1214 is substantially flush with the second surface 1212 of the base 121. However, it is preferred that the cross bar 1214 is configured to be retracted a suitable distance from the second surface 1212, to provide a pivoting space for a guide-engaging element 1215 (described later) in a portion of the interior cavity of the cylindrical anchor alignment member 1213 on the side of the second surface 1212.

Here, the cross bar 1214 is described as being disposed within a cavity (hole) defined by a cylindrical anchor alignment member. However, in the case where the cylindrical anchor alignment member 1213 is fixed to the first surface 1211 of the base 121 in alignment with the through hole 1219 provided in advance in the base 121, the cross bar 1214 may also be provided on the hole wall of the through hole 1219 provided in advance in the base 121. In addition, FIGS. 2A and 2B illustrate a circular through hole 1219, but the through hole 1219 may also take other shapes, such as oval, square, and the like.

In FIG. 2B, the cross bar 1214 is a square bar in cross section, but may also be a circular bar in cross section. The cross bar 1214 may be linear or curved. In addition, the cross bar 1214 is fixedly connected at both ends (e.g., by welding) to the cylinder wall of the cylindrical anchor alignment member. However, the cross bar 1214 may also be fixedly connected at only one end to the cylinder wall of the cylindrical anchor alignment member.

Continuing to refer to FIGS. 2A, 2B and with reference to FIGS. 5 and 6, a guide-engaging element 1215 is further provided at the position of the connection portion 150 of the base 121. In this example, the guide-engaging element 1215 is cylindrical, and includes a large-diameter cylindrical portion and a small-diameter cylindrical portion. The large-diameter cylindrical portion is provided with an axial inner hole 1215S for engaging with the external thread of the external thread portion 1001S of the guide 1001. The small-diameter cylindrical portion is provided with a radial through hole 1215R through which the guide-engaging element 1215 is connected to the cross bar 1214 so as to rotate about the axis of the cross bar.

In FIG. 5, the guide-engaging element 1215 is a cylinder including two different diameter portions, but may also be a cylinder having the same diameter. In addition, the guide-engaging element 1215 in FIG. 5 has a circular cross section, but may also have a cross section of other shapes, such as a square or the like. But preferably the guide-engaging element 1215 has a circular cross section to facilitate rotation of the tissue anchoring element 50 about the guide-engaging element 1215 to anchor the base 121 to the annulus tissue.

Continuing to refer to FIG. 2B, at least one notch 1216 aligned with the guide-engaging element 1215 is provided on the cylinder wall of the cylindrical anchor alignment member 1213. The notch 1216 may be provided at one or both sides of the cross bar 1214 and opens at an end of the cylinder wall of the anchor alignment member that is opposite to the first surface 1211, to receive the guide-engaging element when the guide-engaging element 1215 is tilted.

When the shaping ring 1 according to the present disclosure is delivered into the body through a catheter, the guide 1001 and the guide-engaging element 1215 are connected in advance by a detachable structure, such as a screw connection. Since the guide-engaging element 1215 is connected to the cross bar 1214 through the radial through hole 1215R, and can be pivoted and received in the notch 1216, the guide 1001 can be laid flat for delivery when the shaping ring 1 is loaded into the catheter.

In addition, as shown in FIG. 2A, the cross bar 1214 is provided with a limiting portion configured to align the guide-engaging element 1215 with the notch 1216. For example, the limiting portion may include a limiting block 12141 fixedly disposed at one end of the cross bar 1214 and a limiting ring 12142 detachably disposed at the other end of the cross bar 1214. Prior to welding the cross bar 1214 to the through hole of the base 121, which through hole may alternatively be defined by the cylinder of the cylindrical alignment member, the guide-engaging element 1215 is sleeved on the cross bar 1214, and then the limiting ring 12142 is sleeved on the cross bar 1214. Thereafter, the cross bar 1214 may be welded to the through hole 1219 of the base 121. The limiting block 12141 and the limiting ring 12142 should be sized to ensure that the guide-engaging element 1215 is aligned with the notch 1216.

Alternatively, two limiting rings 12142 may be provided. For example, the limiting rings 12142 may be sleeved on both ends of the cross bar. Then two ends of the cross bar are fixed directly or indirectly in the through hole of the base. By providing limiting blocks and limiting rings on the cross bar, the guide-engaging element is enabled to rotate about the axis of the cross bar at an intermediate position of the cross bar 1214 (i.e., aligned with the notch 1216).

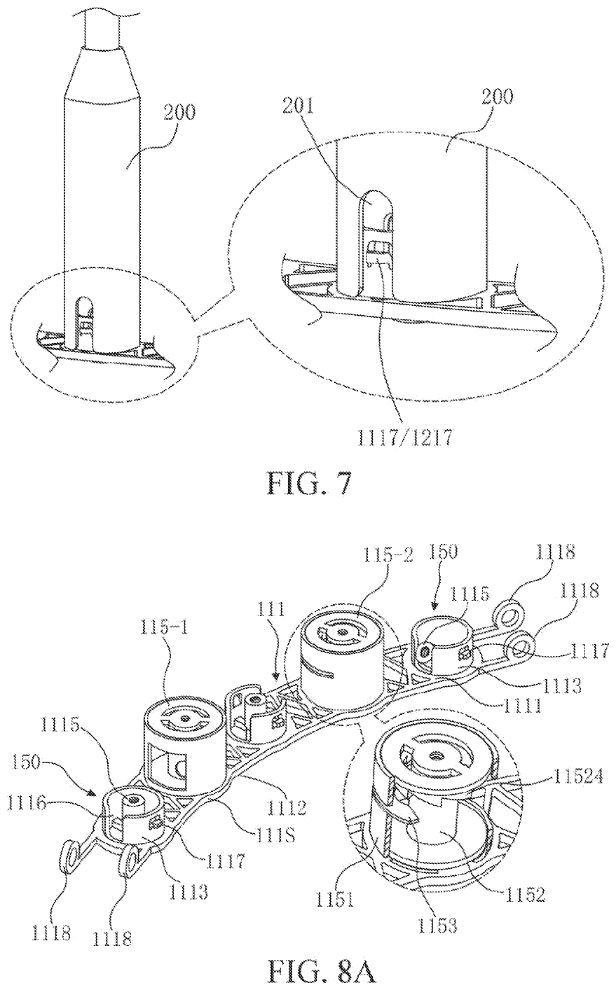

Continuing to refer to FIG. 2B and refer to FIG. 7, at least one projection 1217 (an example of a position adjustment structure of the bar member) is provided on the outer surface of the cylinder wall of the cylindrical anchor alignment member 1213. The projection 1217 may engage with the open slot 201 at a lower end of the torque drive device 200, to adjust the position and/or orientation of the base 121, and in turn adjust the position and/or orientation of the bar member 12 and or the bar member 13. The position and/or orientation of the bar member 12 and/or the bar member 13 may be adjusted by rotating the torque drive device 200 clockwise or counterclockwise prior to anchoring the bar member 12 and/or the bar member 13 to the annulus tissue. The number of the projection 1217 may be the same as the number of the open slot 201.

FIG. 2B illustrates the position adjustment structure as a projection 1217, but the position adjustment structure may take other forms. For example, the cylindrical alignment member may be arranged to have a cross section of an oval, a square, or a rectangle or other shapes. The position and/or orientation of the bar member is thereby adjusted by engagement of the cylindrical alignment member with the torque drive device 200 having a correspondingly shaped distal end.

As shown in FIG. 2A, the base 121 also has a loop 1218 projecting from the circumferential side of the base 121, for connecting the respective extendable and compressible element or interconnecting element. In FIG. 2A, each end of the base 121 is provided with one loop 1218, and two or more loops 1218 may be provided as well. The loop 1218 may be a completely closed loop, or may be a partially closed or partially open loop. The present disclosure has no limitation to the specific structure of the loop as long as the fixed connection between the base and the extendable and compressible element can be achieved. In addition, the loop 1218 may be a circular loop, or may be a loop of other shapes, such as an oval shape. FIGS. 3A and 3B also show two examples of loops that are not completely closed.

FIG. 4 shows one example of a known tissue anchoring element 50 for connecting the bar members 11, 12, 13 of an annuloplasty device of the present disclosure to the annulus tissue. In this example, the tissue anchoring element 50 is a helical tissue anchoring element that includes a head portion 501 and a helical portion 502 having a sharpened distal tip. The head portion 501 is provided with an axial through hole 5011 through which the guide 1001 passes, and a proximal end of the helical portion 502 is fixed to the head portion 501. After the helical tissue anchoring element 50 is securely anchored into the annulus tissue, the head portion 501 is pressed against the respective cross bar. The connection of the bar members 11 to 13 to the annulus tissue by a helical tissue anchoring element 50 is described herein. However, other types of tissue anchoring elements may be employed as long as the tissue anchoring element is capable of anchoring the bar members 11 to 13 to the annulus tissue in cooperation with the cross bars 1114, 1214 when the tissue anchoring element is anchored to the annulus tissue.

FIG. 6 shows a guide 1001 used in the guiding and anchoring of the shaping ring 1 of the present disclosure. The guide-engaging element 1215 is provided with a structure that is detachably connected with the guide 1001. Referring to FIG. 5, the guide-engaging element 1215 is provided with an internal thread 1215S and the guide member 1001 is provided with an external thread 1001S, both of which are threadedly connected to detachably connect the guide-engaging element 1215 and the guide 1001 together.

FIG. 7 schematically shows a torque drive device 200 for adjusting the position and/or orientation of the bar member 12 or 13. As shown, the open slot 201 of the torque drive device 200 engages with a projection 1217 provided on the cylinder wall of the anchor alignment member 1213, to thereby adjust the position and/or orientation of the bar member 12 or 13. The torque drive device 200 may likewise engage with a projection 1117 on the cylinder wall of the cylindrical anchor alignment member 1113 provided on the base 111 of the bar member 11, to thereby adjust the position and/or orientation of the bar member 11.

Hereinafter, the bar member 11 will be described with reference to FIGS. 8A and 8B. The bar member 11 includes a flat plate-shaped base 111. In the example shown in FIG. 8A, the base 111 is an elongated base substantially equal to the arch length of the posterior leaflet P2 (see FIG. 18) of the mitral valve. Of course, the base 111 may also take any other suitable shape, and the length of the base 111 may vary as the case may be, so long as it allows the desired leaflet portions to be brought close to each other without any leaflet crinkling, and in addition, mitral regurgitation can be reduced or eliminated.

The base 111 includes a first surface 1111, a second surface 1112, and a circumferential side 111S connecting the first surface 1111 and the second surface 1112. The base 111 also includes a plurality of through holes (e.g., triangular and quadrangular through holes as shown in the drawings) through the base, but the base 111 may not include these through holes, but a complete solid base. The flexibility of the base can be improved by providing through holes.

In addition, the base 111 may be provided with at least one (three shown in FIG. 8A) connection portion 150 at which a cylindrical anchor alignment member 1113, a cross bar 1114, and a guide-engaging element 1115 are provided. Similar to the anchor alignment member 1213, the cylinder wall of anchor alignment member 1113 is provided with a notch 1116 and the outer surface of the cylinder wall is provided with at least one projection 1117. The structure and operation of the connection portion 150 (including the cylindrical anchor alignment member 1113, the cross bar 1114, the guide-engaging element 1115) of the bar member 11 are the same as that of the connection portion 150 (including the cylindrical anchor alignment member 1213, the cross bar 1214, the guide-engaging element 1215) of the bar member 12, 13, and will not be described in detail herein.

The cross bars 1214, 1114 act as abutments for the connection portion 150 and the head portion 501 is pressed against the respective cross bar after the helical tissue anchoring element 50 is securely anchored into the annulus tissue. It should be noted that examples of the abutments are not limited to the cross bars 1214, 1114 as shown in FIGS. 2A and 8B, and may take other forms or shapes, as long as the helical portion 502 of the tissue anchoring element 50 is able to fix the bar members 11, 12, 13 to the annulus tissue through the abutment. For example, the abutment may also take the form of a fan-shaped suspending wall (not shown) extending beyond the center of the through hole 1119 inwardly from the wall of the through hole 1119 and substantially parallel to the first surface 1111 of the base 111. The fan-shaped suspending wall occupies a portion of the through hole 1119 in a circumferential direction of the wall of the through hole 1119 to define an opening between the fan-shaped suspending wall and the wall of the through hole 1119, which opening allows the helical portion 502 of the spiral tissue anchoring element 50 to pass through. The fan-shaped suspending wall may be located at any position on the wall of the through hole 1119 along the axis of the through hole 1119. Further, a threaded hole, which is used for detachably connecting with the external thread 1001S of the distal end of the guide 1001, may be provided at a portion of the fan-shaped suspending wall, which portion is located at the center of the through hole 1119. The fan-shaped suspending wall may occupy ⅙ to ⅝ or other percentage of the circumference of the through hole 1119, as long as the opening between the fan-shaped suspending wall and the wall of the through hole 1119 can allow the helical portion 502 to pass through. Similarly, the fan-shaped suspending wall structure of the bar member 11 can also be applied for the bar members 12, 13, which will not be described here.

As shown in FIG. 8A, each end of the base 111 is also provided with a loop 1118 for connecting the extendable and compressible elements 14, 16. Two loops 1118 may be provided at each end of the base 111 to provide a more secure connection of the extendable and compressible element to the base 111. In addition, it is preferable that the center distance of the outer two loops 1118 is larger than the center distance of the inner two loops 1118, so as to form a natural transition ring shape after the bar member 11 is connected to the extendable and compressible element. The loop 1118 may be identical to the loop 1218 of the bar members 12, 13.

In the embodiment shown in FIG. 1A, the base 111 is provided with two cinching devices 115-1, 115-2. The reels of the cinching devices 115-1, 115-2 are each rotatable about an axis perpendicular to the first surface 1111 of the base 111. When two cinching devices are provided, the adjustment wire 171 and the adjustment wire 172 can be independently adjusted, respectively, thereby more advantageously adjusting the shape of the annulus. For example, when different regions of the physiological annulus require different amounts of contraction, it is advantageous to provide an embodiment with two cinching devices 115-1, 115-2.

In another example, the base 111 may also be provided with only one cinching device. In this case, both the adjustment wire 171 and the adjustment wire 172 are connected to the cinching device. When the reel of the cinching device is rotated, the adjustment wire 171 and the adjustment wire 172 are simultaneously tightened or released. The advantage of this example is that the two adjustment wires can be adjusted at the same time, and the adjustment efficiency is improved.

The cinching devices 115-1, 115-2 can be in an unlocked state or a locked state. When the cinching devices 115-1, 115-2 are in an unlocked state, the reels of the cinching devices may be driven to rotate, and when in a locked state, the reels of the cinching devices are locked in the direction of releasing the adjustment wire so as not to be rotatable in the direction of releasing the adjustment wire tension, thereby maintaining the adjustment line in tension.

FIG. 1A shows a case where both of the cinching devices 115-1, 115-2 are provided on the bar member 11, but the present disclosure is not limited thereto. For example, the cinching device 115-1 may be provided at the bar member 12, and the cinching device 115-2 may be provided at the bar member 13. Alternatively, the cinching device 115-1 may be provided at the bar member 11, and the cinching device 115-2 may be provided at any one of the bar member 12 and the bar member 13. Those skilled in the art, under the teachings of the present disclosure, will be able to provide the cinching device 115 on the appropriate bar members 11, 12, 13, thereby achieving the contraction of the annulus.

In addition, the embodiment of FIG. 1A employs two adjustment wires 171 and 172. However, only one adjustment wire may be used. For example, one end of an adjustment wire extends through the extendable and compressible element 14 to be fixed to the bar member 12 and the other end extends through the extendable and compressible element 16 to be connected to the bar member 13. In this case, each of the bar member 12 and the bar member 13 may be provided with a cinching device.

It has been described above that the tension of the adjustment wire is adjusted by providing a rotatable cinching device. However, the adjustment may also be implemented in other existing ways. For example, after the desired valve annulus size has been obtained, the adjustment wires 171, 172 may be connected to the respective bar members 11, 12, 13 and fixed by means of such as knotting. In some embodiments, a reversible lock may be used during the cinching process, and the reversible lock may be configured to permanently maintain the position of the adjustment wire. The present disclosure preferably uses rotatable cinching devices (e.g., the cinching devices 115-1, 115-2) for contraction of the extendable and compressible elements and fixation of the adjustment wires.

Other suitable arrangement locations and number of arrangements of the cinching device and the connection portion will readily occur to those skilled in the art under the teachings of the present disclosure.

FIG. 1A shows an example in which the extendable and compressible elements 14 and 16 and the interconnecting element 15 are three individual coil elements. However, the present disclosure is not limited thereto. For example, in the case where the interconnecting element 15 is provided between the bar member 12 and the bar member 13, the extendable and compressible elements 14 and 16 and the interconnecting element 15 may be constituted by a single coil element 400. As shown in FIG. 1B, the single coil element 400 sequentially connects the bar members 11, 12, 13 by sleeving respective coils on the cylindrical anchor alignment members of the respective bar members 11, 12, 13, wherein the bar member 11 are connected to both ends of the coil element 400, and the bar members 12 and 13 are connected to the coil element 400 between both ends of the coil element 400. Alternatively, in the case where the interconnecting member 15 is not provided between the bar member 12 and the bar member 13, one end of the coil element 400 may be connected to the bar member 12 and the other end connected to the bar member 13 via the bar member 11. Likewise, the coil element 400 may be connected to the respective bar member by sleeving the coil on the cylindrical anchor alignment member of the respective bar member. Alternatively or additionally, the respective coil of the coil element 400 is welded to the respective bar member (e.g., the anchor alignment member). In the example shown in FIG. 1B, the bar members 11 to 13 may not be provided with loops 1118, 1218. In the example of FIG. 1A, FIG. 1i, or the like, since three bar members 11, 12, and 13 are provided, the physiological annulus can be linearly reduced in the direction of the AP axis. In addition, since the separately arranged bar members are connected by using the extendable and compressible elements, it is possible to selectively adjust the size of the physiological valve annulus at a specific part without having to adjust the valve annulus as a whole.

Alternatively or preferably, the bar member 11 may be folded, such as the bar member 11′ shown in FIGS. 8C and 8D, which includes two sub bar members 11′-1, 11′-2, wherein the sub bar members 11′-1, 11′-2 may be hinged to each other, such that they can folded over each other. For example, the bar member 11′ may include a hinge portion 113, wherein the hinge portion 113 may include a ring-shaped member 1119 to which a loop 1118 at one of the ends of the sub bar members 11′-1 and 11′-2 is connected, and folding of the bar member 11′ is effected by rotation or sliding of the loop 1118 about the ring-shaped member 1119. The ring-shaped member 1119 may be circular, oval or irregular polygonal in shape. Alternatively, the hinge portion 113 may include an elongated shaft (not shown) passing alternately through the loops 1118 of the sub bar members 11′-1 and 11′-2, in such a way that the sub bar members 11′-1 and 11′-2 can be folded over each other by means of the pin shaft. Folding the bar member may help load the shaping ring into the catheter and facilitate manipulation in the left atrium.

FIGS. 8C and 8D show that the bar member 11′ comprises two sub bar members 11′-1, 11′-2. However, the bar member 11′ may include three or more sub bar members, which may be connected to each other by respective hinge portion 113 so that they may be folded over each other.

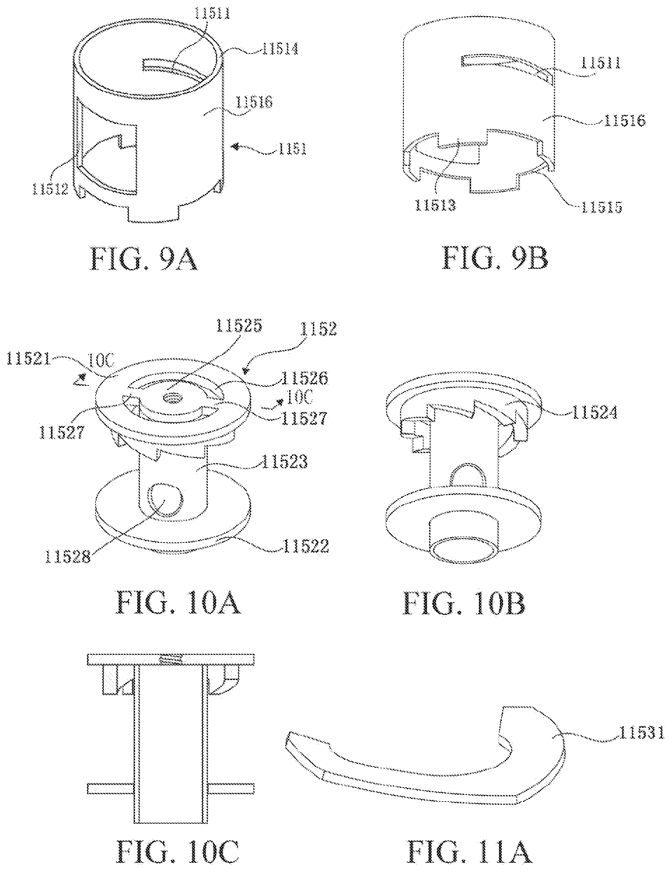

Hereinafter the structure of the cinching devices 115-1 and 115-2 will be described in detail with reference to FIGS. 9A to 13C. It should be noted that the structures of the cinching devices 115-1, 115-2 and other cinching devices mentioned below and/or shown in the drawings are the same, and only the cinching device 115-1 will be described as an example.

As shown in FIGS. 8A, 9A to 11D, the cinching device 115-1 includes a housing 1151, a reel 1152, and an elastic sheet 1153. The housing 1151 defines an upper surface 11514, a lower surface 11515, and a sidewall 11516 extending between the upper surface and the lower surface. A slot 11511 is provided on the sidewall 11516 of the housing, for fixedly placing and connecting the elastic sheet 1153. The position and orientation of the slot 11511 may be appropriately determined depending on the shape and placement position of the fixed side 11531 of the elastic sheet 1153 employed.

The side wall 11516 is provided with a hole 11512 into which the adjustment wire penetrates. A square hole 11512 is shown in FIG. 9A, but the hole 11512 may be any other shape, such as a circular hole, a polygonal hole, or the like. The lower surface 11515 of the housing 1151 is projectingly provided with a plurality of spaced apart flanges 11513. The flange 11513 is configured to be inserted into a corresponding groove 11120 provided in the base 111 of the bar member 11 (see FIG. 8B), to fix the housing 1151 to the base 111. The number and shape of the groove 11120 in the base 111 corresponds to the number and shape of the flange 11513. If the cinching device 115 is provided on the bar member 12 and/or 13, the base 121 of the bar member 12 and/or 13 may also be similarly provided with a groove (not shown) for engagement with the flange 11513. The placement position and shape of such groove of the base 121 may be the same as the groove 11120. Preferably, after the flange 11513 of the housing 1151 is inserted into the groove 11120 of the base 111, the flange 11513 and the groove 11120 are fixed by welding.

FIGS. 10A to 10C show a reel 1152. The reel 1152 may be rotatably disposed within the housing 1151. The reel 1152 includes an upper plate 11521, a lower plate 11522, a reel body 11523 extending between the upper and lower plates, and ratchet teeth 11524. The upper plate 11521 is fixedly connected to the reel body 11523 and is provided with a screw hole 11525 for being detachably connected with the external thread 1001S of the guide 1001. The ratchet teeth 11524 are provided on the lower surface of the upper plate 11521 around the reel body 11523 and spaced apart from the reel body 11523. The upper plate 11521 is provided with at least one torque drive device engaging feature 11527 configured to engage with a drive feature (e.g., an open slot 301) of the torque drive device 300. In the example shown in FIGS. 10A to 10C, the torque drive device engaging feature is a rib portion defined between two arcuate slots 11526 extending through the upper plate.

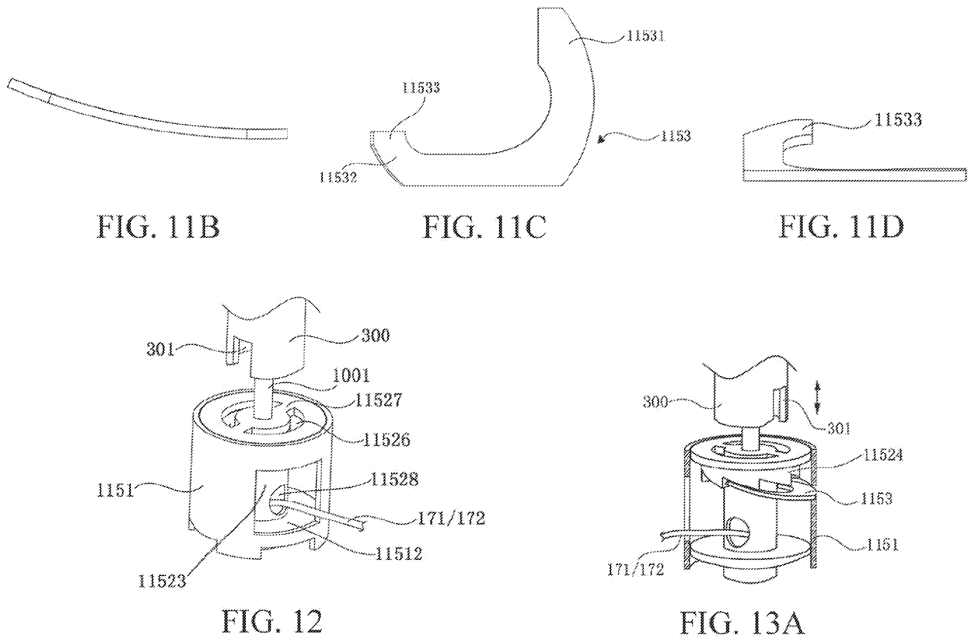

Referring to FIGS. 11A to 11D, the elastic sheet 1153 has a generally L-shape and includes a fixing side 11531 for being fixed to the slot 11511 of the housing 1151 and a ratchet engaging side 11532 for being engaged with the ratchet 11524, wherein the ratchet engaging side 11532 includes a protrusion 11533 that extends into the space between the ratchet teeth 11524 and the reel body 11523.

Referring to FIG. 12, the torque drive device 300 may have a cylindrical shape with a distal end side provided with an open slot 301 that may engage with the engaging feature 11527 to drive the upper plate 11521 and thereby drive the rotation of the reel 1152. The torque drive device 300 may be engaged with the protrusion 11533 of the ratchet engaging side 11532 of the elastic sheet 1153 by extending through the arc-shaped through groove 11526 downward by means of the open slot 301, thereby the elastic sheet 1153 is disengaged from the ratchet teeth 11524 provided on the lower surface of the upper plate 11521. The depth of the open slot 300 should ensure that the torque drive device 300 can sufficiently disengages the elastic sheet 1153 from the ratchet teeth 11524, but also prevents the elastic sheet 1153 from over-flexing downward and losing the shape recovery capability.

In FIG. 10A, the upper plate 11521 is provided with two torque drive device engaging features 11527. However, the upper plate 11521 may be provided with more than two torque drive device engaging features 11527. Accordingly, the torque drive device 300 may be provided with the same or fewer number of open slots 301 as the number of engaging features 11527.

The reel 1152 may be rotatably fixed into the through hole 1120 of the base 111 of the bar member 11, and the lower plate 11522 of the reel 1152 may snugly abut against the first surface 1111 of the base 111 (see FIG. 8B).

When the ratchet engaging side 11532 of the elastic sheet 1153 is engaged with the ratchet teeth 11524 of the reel 1152, the reel 1152 can only be driven in one-way rotation, prohibiting reverse rotation, and the cinching device is in the one-way locking state. When the torque drive device 300 moves downward to contact with the protrusion 11533 of the elastic sheet 1153 and further presses the protrusion 11533 downward, the elastic sheet 1153 is separated from the ratchet teeth 11524 of the reel 1152, and the reel can be driven to rotate bi-directionally, and the cinching device is in an unlocked state.

FIGS. 10A to 10C show that the reel 1152 has one-way ratchet teeth. However, the reel 1152 may have bi-directional ratchet teeth, such as square teeth. In this case, when the elastic sheet 1153 abuts against the ratchet teeth 11524 of the reel 1152, the cinching device is locked in a locked state in both directions, and the unlocked state coincides with the one-way ratchet teeth.

As shown in FIG. 12, one end of an adjustment wire (e.g., the adjustment wire 171) is inserted into an adjustment wire hole 11528 of the reel body 11523 via a hole 11512 of the housing 1151 (see FIG. 10A), and may be connected with the reel body 11523 by knotting or other means. The other end of the adjustment wire is fixed to the corresponding bar member. For example, in the embodiment of FIG. 1A, one end of the adjustment wire 171 is connected to the reel body 11523, and the other end is connected to the bar member 12. The adjustment wire 172 is connected in the same manner as the adjustment wire 171.

As described above, the upper plate 11521 of the reel 1152 is provided with features 11527 that engage with the torque drive device 300. The torque drive device 300 releases the locked state of the reel 1152 by pressing the protrusion 11533 of the elastic sheet 1153 downward to disengage the ratchet engaging side 11532 of the elastic sheet 1153 from the ratchet teeth 11524 of the reel 1152. At this time, since the open slot 301 of the torque drive device 300 engages on the engaging feature 11527, as the torque drive device 300 rotates, the reel 1152 rotates towards the tightening direction, an adjustment wire, such as the adjustment wire 171, may be wound around the reel body 11523 of the reel 1152 to achieve contraction of the annulus. If the annulus is adjusted too small, the torque drive device 300 may be rotated in a release direction opposite to the tightening direction, the reel 1152 is rotated in the opposite release direction, thereby unwinding the adjustment wire (e.g., adjustment wire 171), so as to be able to make the annulus become larger again. If the size of the annulus is adjusted to an appropriate size, the torque drive device 300 is disengaged from the protrusion 11533 of the elastic sheet 1153 and the elastic sheet 1153 is released, the ratchet engaging side 11532 of the elastic sheet 1153 thus engages the ratchet teeth 11524 to lock the reel 1152 in place.

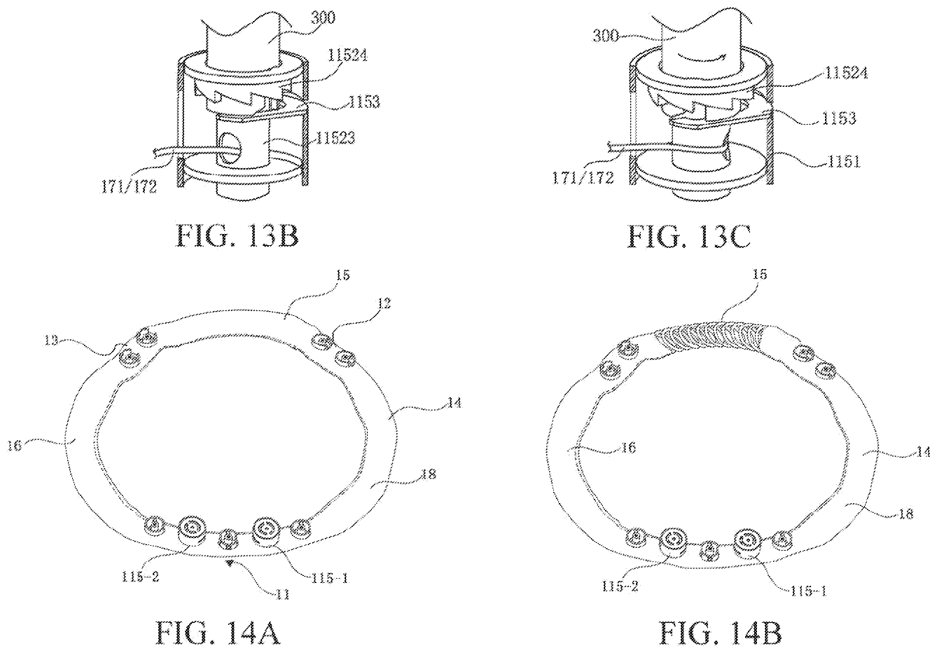

FIGS. 13A to 13C illustrate one example of an adjustment process of the cinching device.

As shown in FIG. 13A, the ratchet engaging side 11532 of the elastic sheet 1153 abuts against the one-way ratchet teeth 11524 of the reel 1152, so that the cinching device is in a one-way locked state, and the elastic sheet 1153 prevents the reel 1152 from rotating counterclockwise (in the unwinding direction); at this time, the torque drive device 300 can drive the reel 1152 to rotate in the clockwise direction (the cinching direction) without releasing the one-way locked state. As shown in FIG. 13B, the torque drive device 300 moves downward through the arc-shaped through groove 11526 in the upper plate 11521 to separate the elastic sheet 1153 from the one-way ratchet teeth 11524, and the cinching device is in an unlocked state. When the cinching device is in an unlocked state, the torque drive device 300 may drive the reel 1152 to rotate clockwise or counterclockwise, thereby properly adjusting the adjustment wire, and thereby adjusting the amount of contraction of the extendable and compressible elements (e.g. the extendable and compressible elements 14, 16 in FIG. 1A) associated with the adjustment wire. FIG. 13C shows an example in which the cinching device, when in the unlocked state, drives the reel to rotate in a counterclockwise direction to unwind and release the adjustment wire.

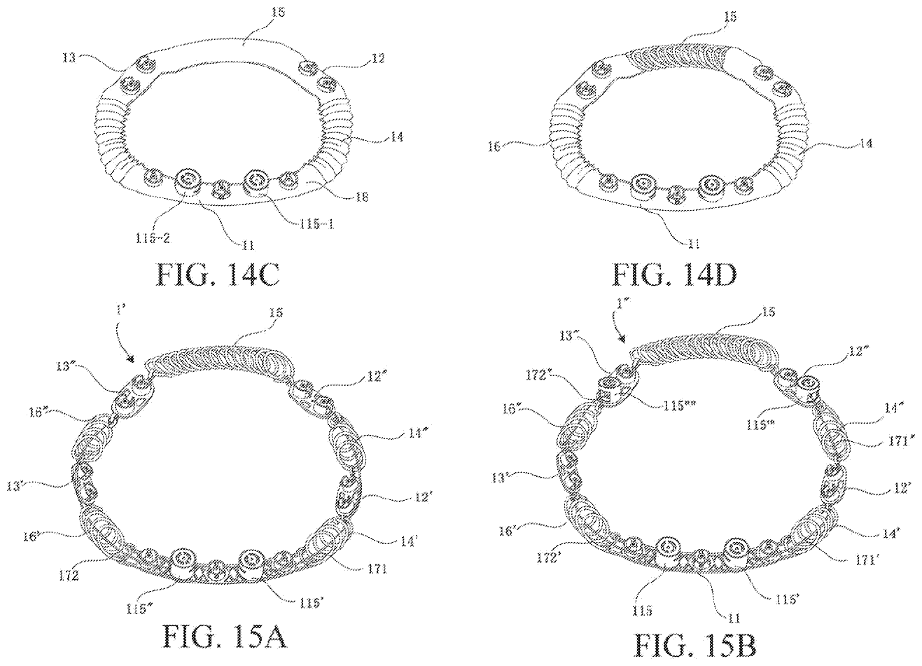

Hereinafter the description of the annuloplasty device including the shaping ring 1 corresponding to FIG. 1A will be continued with reference to FIGS. 14A to 14D.

As shown in FIG. 14A, the shaping ring 1 may also comprise a fabric 18 which partially covers the shaping ring 1 to expose the cinching device and the anchor alignment member provided at the bar member.

FIGS. 14A and 14B show the shaping ring 1 in a naturally expanded state, and FIGS. 14C and 14D show the shaping ring 1 in a contracted state after being adjusted. As can be seen from FIGS. 14B and 14D, the optional interconnecting element 15 is substantially in the naturally extended state when the shaping ring 1 is in both the naturally expanded state and the contracted state. This is because no adjustment wire is provided in the inner cavity of the interconnecting element 15.

In addition, both the extendable and compressible elements 14, 16 and the optional interconnecting element 15 may employ inclined coil elements, wherein the coils of the coil elements may be configured to be inclined with respect to the longitudinal centerline of the respective extendable and compressible elements 14, 16 or of the optional interconnecting element 15 so that the extendable and compressible elements or the interconnecting element can snugly abut against the surface of the annulus tissue. Of course, the extendable and compressible element may also take the form of an elastic extendable and compressible element other than a coil element, as long as it has an elastic property of being stretchable and contractible.

For the shaping ring 1 shown in FIG. 1A or FIG. 14A, all the constituent elements (such as the bar members 11 to 13, the extendable and compressible elements 14, 16, the optional interconnecting element 15, the cinching devices 115-1, 115-2 and the adjustment wires 171, 172) of the shaping ring 1 shown in FIG. 1A can be assembled in advance in vitro. Thus, after the shaping ring 1 is delivered into the body, the surgical procedure involves only the anchoring of the bar member and the tensioning of the adjustment wire, thereby making the surgical procedure simple and reliable and reducing the surgical operation time in the body. In particular, when the exemplary shaping ring 1 shown in FIG. 1A is implanted in the body, the central connection portion of the bar member 11 may be fixed to the vicinity or center of the P2 annulus of the mitral annulus tissue by a tissue anchoring element (such as a helical tissue anchoring element as shown in FIG. 4). The connection portion of the bar member 12 is fixed to the medialtrigone region of the mitral annulus tissue, and the connection portion of the bar member 13 is fixed to the lateral trigone region of the mitral annulus tissue. In this way, when the shaping ring 1 is in the contracted state, the distance in the direction of the AP axis can be significantly reduced, thereby bringing the anterior and posterior leaflets closer together to effectively reduce regurgitation. In addition, in this embodiment, as shown in FIGS. 2A and 8B, the bar member 11 is provided with three connection portions and two cinching devices. The bar members 12 and 13 have the same structure and are both provided with two connection portions. Of course, FIG. 1A illustrates merely an example. The number of the connection portions of the bar members 11, 12, 13 and on which bar member the cinching device is arranged can be appropriately adjusted according to the actual application.

In a preferred example, at least two ends of the bar member 11 may be anchored to the region P2 (or its vicinity) of the posterior leaflet of the annulus tissue by respective connection portion via the tissue anchoring element. By anchoring the bar member 11 to the P2 annulus at both ends of the bar member 11, it is possible to ensure that the length of the physiological valve annulus in the P2 region is substantially unchanged during the adjustment process, and to avoid wrinkling of the normally functioning posterior leaflet, thereby preventing the occurrence of new regurgitation due to the annulus contraction.

It should also be noted that FIGS. 1A, 14A to 14D only exemplarily show the anchoring position of the shaping ring 1 of the present disclosure. In the use of the shaping ring of the present disclosure, the specific number of bar member 11, the bar member 12 and the bar member 13 and the anchoring positions of the connection portions can be adjusted as appropriate in accordance with the teachings of the present disclosure in a specific case of poor closure of the mitral valve. The correction of the tricuspid valve may be performed similarly to the correction of the mitral valve.

It should be noted that the annuloplasty device including the shaping ring corresponding to FIG. 1B is similar to the annuloplasty device including the shaping ring corresponding to FIG. 1A, and will not be described in detail herein.

FIG. 15A shows a shaping ring 1′ in accordance with another embodiment of the present disclosure. The shaping ring 1′ differs from the shaping ring 1 in that the shaping ring 1′ comprises two bar members 12′, 12″ and two bar members 13′, 13″. In FIG. 15A, the bar members 12′, 12″ and the bar members 13′, 13″ are identical to the bar members 12 and 13. However, they may differ from one another, for example with a different number of connection portions or with or without a cinching device.

Moreover, the difference also lies in that the shaping ring 1′ comprises two extendable and compressible elements 14′, 14″ and two extendable and compressible elements 16′ and 16″. The shaping ring 1′ uses segmented extendable and compressible elements and thus two further bar members 12′ and 13′. One end of the adjustment wire 171 is connected to the cinching device 115′, and the other end extends through the extendable and compressible elements 14′, 14″ and is connected to the bar member 12″. One end of the adjustment wire 172 is connected to the cinching device 115″, and the other end extends through the extendable and compressible elements 16′, 16″ and is connected to the bar member 13″.

Other aspects of the shaping ring 1′ shown in FIG. 15A are substantially the same as those of the shaping ring 1 shown in FIG. 1A. In addition, the structure and mode of function of the cinching devices 115′, 115″ are the same as the cinching device 115-1. In FIG. 15A, the cinching devices 115′ and 115″ may also be provided on the bar members 12′ and 13′ instead of on the bar member 11. The cinching device may also be arranged on other bar members, and a person skilled in the art may arrange the cinching device on a suitable bar member under the teachings of the present disclosure.

In the example shown in FIG. 15A, there is no adjustment wire inside the optional interconnecting element 15. In addition, both the extendable and compressible elements 14′, 14″, 16′, 16″ and the optional interconnecting element 15 may be coil elements, wherein the coils of the coil elements may be configured to be inclined with respect to the longitudinal centerline of the respective extendable and compressible elements or of the optional element so that the extendable and compressible elements or the interconnecting element can snugly abut against the surface of the annulus tissue.

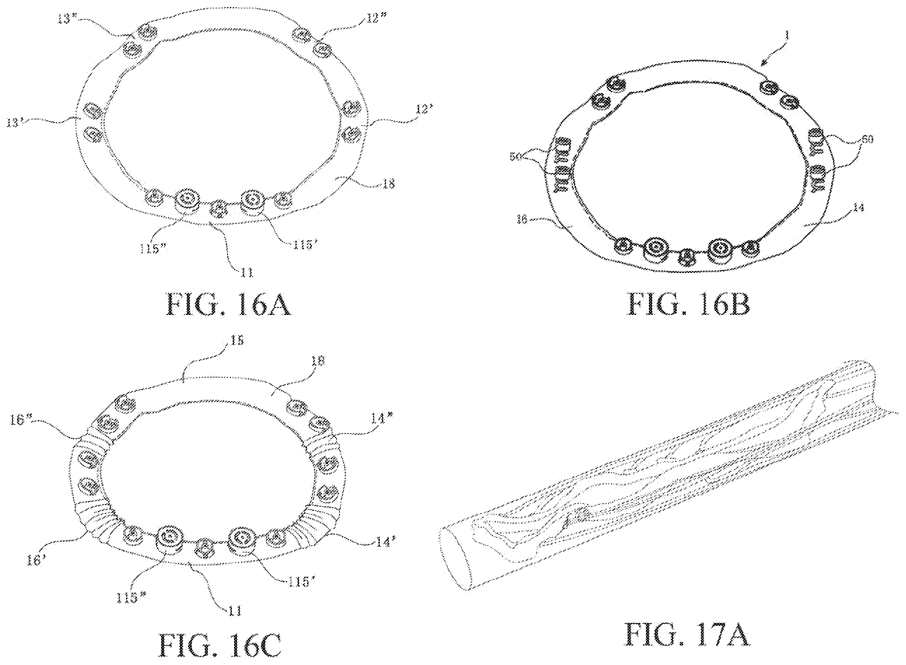

FIG. 16A shows the shaping ring 1′ covered with the fabrics 18, and FIG. 16C shows the shaping ring 1′ after the predetermined contraction is performed.

FIG. 15B shows a shaping ring 1″ in accordance with another embodiment of the present disclosure. The shaping ring 1″ differs from the shaping ring 1′ in that the first adjustment wire 171 is replaced by two adjustment wires 171′ and 171″ and the second adjustment wire 172 is replaced by 172′ and 172″ and the bar member 12″ is provided with a cinching device 115′″, and the bar member 13″ is provided with a cinching device 115″″. One end of the adjustment wire 171′ is connected to the cinching device 115′ provided at the bar member 11, and the other end extends through the extendable and compressible element 14′ and is connected to the bar member 12′. The adjustment wires 171″, 172′ and 172″ are arranged in a manner similar to the adjustment wire 171′, each being connected to a respective cinching device and extending through a respective extendable and compressible element to be coupled to another bar member.

If the bar member 12″ is provided with a cinching device 115′″, the portion of the base 121 of the bar member 12″ where the cinching device 115′″ is provided may be provided with the same structure as the portion of the base 111 of the bar member 11 where the cinching device 115-1, 115-2 are provided. It should be noted that all of the cinching devices 115-1, 115-2, 115′, 115″, 115′″, 115″″ mentioned herein have the same structure and mode of function.

In FIG. 15B, the cinching devices 115′″ and 115″″ may also be provided on the bar members 12′ and 13′, respectively. The shaping ring 1″ shown in FIG. 15B can achieve the same adjustment effect as the shaping ring 1′ of FIG. 15A.

The implantation process of the annuloplasty device comprising the shaping rings 1′, 1″ shown in FIGS. 15A and 15B is similar to that of the annuloplasty device comprising the shaping ring 1, and it is simply necessary to anchor the two additional bar members 12′, 13′ at or near the P1 and P3 regions respectively (see FIG. 18).

In the example shown in FIG. 1B, a single extendable and compressible element 400 is used in place of the extendable and compressible elements 14, 16 and the interconnecting element 15. Similarly, in the example shown in FIGS. 15A and 15B, a single extendable and compressible element (such as a coil element) may also be used in place of the extendable and compressible elements 14′, 14″, 16″, 16′ and the interconnecting element 15 (if provided). The connection of the bar members 12′, 12″, 13″, 13′ to the single extendable and compressible element can be achieved by sleeving the coil of the extendable and compressible element around and optionally welded to the anchor alignment member of the respective bar members.

Referring to the shaping rings 1′, 1″ as shown in FIGS. 15A, 15B, since the bar members 11, 12″ and 13″ are provided in the medial and lateral trigone regions, the physiological annulus can be linearly reduced in the direction of the AP axis; and, in turn, by providing the bar members 12′ and 13′ in the regions P1 and P3, the physiological annulus can also be constricted around the anterior commissure and the posterior commissure. In addition, since the separately arranged bar members are connected by using the extendable and compressible element, it is possible to selectively adjust the size of the physiological valve annulus at a specific part without having to adjust the valve annulus as a whole.

Alternatively, as in the example shown in FIG. 16B, at least one conventional helical tissue anchoring element 50 as shown in FIG. 4 is added at the extendable and compressible elements of the shaping ring 1 at the P1 and P3 regions. In this example, the positions of the extendable and compressible elements in the P1 and P3 regions are respectively provided with two helical tissue anchoring elements 50. The annulus can also be constricted circularly in the anterior and posterior commissure regions by the helical tissue anchoring element 50 securing the respective extendable and compressible elements to the annulus tissues at or near the regions P1 and P3.

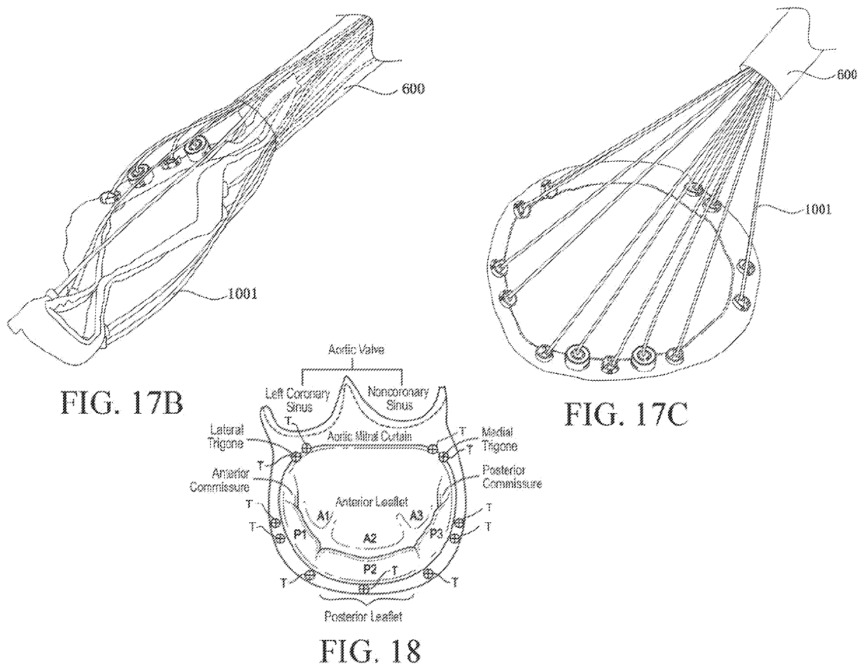

FIG. 17A schematically shows the shaping ring in a folded state and in a catheter, FIG. 17B shows the shaping ring in a partially unfolded state and partially in the catheter, and FIG. 17C shows the shaping ring in a fully unfolded state. Since the extendable and compressible element (e.g., the coil element) can be fitted into the catheter in a folded state, when the shaping ring is deployed from the catheter, the shaping ring can be automatically unfolded from the folded state to the initial naturally expanded state by the stretchability of the extendable and compressible element.

In the shaping rings 1, 1′, 1″ described above, any biocompatible material may be used to manufacture the bar members, the extendable and compressible elements, the optional interconnecting element, the cinching device and/or the adjustment wires of the shaping rings. For example, a biocompatible polymeric material or metallic material may be used.

For example, the adjustment wire may be a filamentous material, a belt, a cord, or a suture. Typically, the adjustment wire comprises a flexible and/or superelastic material, such as Nitinol, polyester, stainless steel, or cobalt chrome alloy, and is configured to be permanently present within the respective extendable and compressible element, such as a flat coil. In some applications, the adjustment wire comprises a woven polyester suture (e.g., Ticron). In some applications, the adjustment wire may be coated with polytetrafluoroethylene (PTFE). In some applications, the adjustment wire comprises a plurality of filamentous materials that are interwoven with each other to form a cord structure. The adjustment wire includes cords or cables that are formed by connecting (e.g., twisting, weaving, or otherwise connecting) threads of a plurality of metals, polymers, or fabrics.

The extendable and compressible element may comprise a biocompatible material such as Nitinol, stainless steel, platinum-iridium alloy, titanium, expanded polytetrafluoroethylene (ePTFE), or cobalt-chromium alloy. The extendable and compressible element may be coated with PTFE (polytetrafluoroethylene). In other applications of the present disclosure, the extendable and compressible element and the optional interconnecting element may be an accordion-like compressible structure that promotes proper tightening of the annulus when the shaping ring is contracted.

The fabrics may employ a polyethylene terephthalate (PET) fabric through which the shaping rings are covered to aid in tissue ingrowth.

The bar members may be a strip shape or may have other suitable shapes. The bar member may be flexible or rigid, preferably with a degree of flexibility to allow the bar member to be adjusted in vivo to fit a particular targeted anatomical structure. The flexibility of the bar member may also allow the bar member to bend during the cardiac cycle. The bar member may comprise a biocompatible material such as Nitinol, stainless steel, platinum-iridium alloy, titanium, expanded polytetrafluoroethylene (ePTFE), or cobalt-chromium alloy. In some applications, the bar member may be coated with polytetrafluoroethylene (PTFE).

FIG. 18 is a general schematic diagram showing a human mitral valve. The mitral valve consists of an anterior leaflet (an anterior valve) and a posterior leaflet (a posterior valve). The anterior leaflet consists of three regions, namely A1, A2 and A3 regions, and the posterior leaflet consists of P1, P2 and P3 regions. Reference sign T in FIG. 18 denotes a possible anchoring position of the connection portion (i.e., the anchoring position of the tissue anchoring element 50). It should be noted that those skilled in the art may select and provide more or fewer anchoring positions and provide a suitable connection member for the anchoring element in accordance with the teaching of the present disclosure.

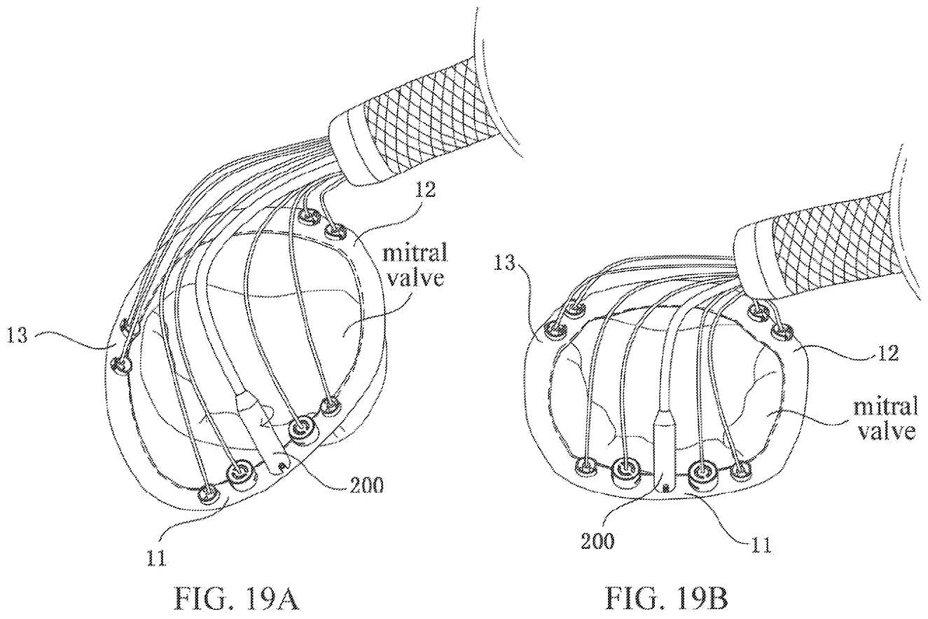

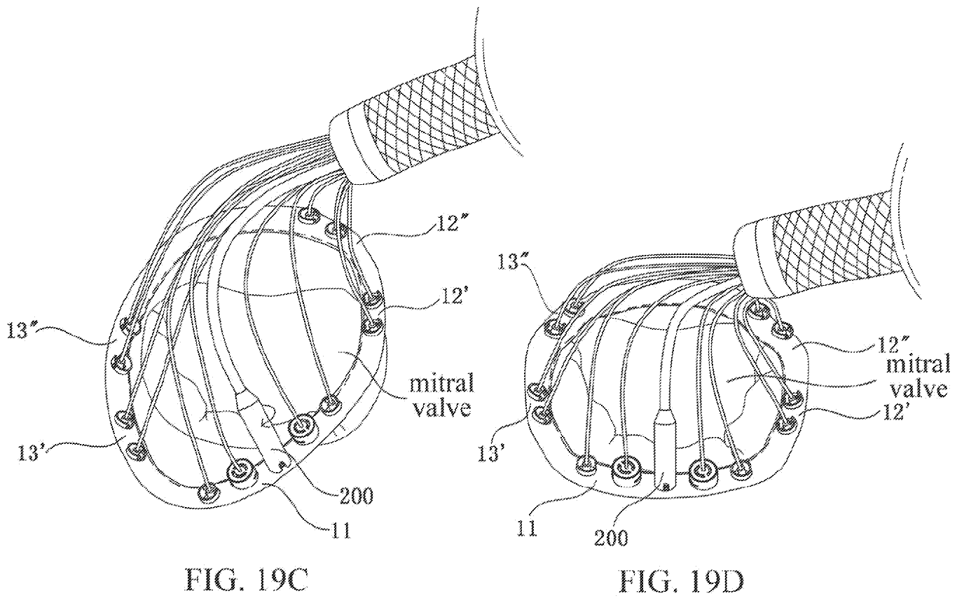

FIGS. 19A and 19B show a schematic diagram of adjusting the position and orientation of, for example, a shaping ring 1 having three bar members to align the shaping ring with a physiological mitral valve by controlling the torque drive device 200 to rotate in the clockwise direction. FIGS. 19C and 19D show a schematic diagram of adjusting the position and orientation of, for example, a shaping ring 1′ or 1″ having five bar members to align the shaping ring with a physiological mitral valve by controlling the torque drive device 200 to rotate in the clockwise direction. It should be noted that the torque drive device 200 may be rotated in the counterclockwise direction to adjust the position and direction of the bar member according to actual needs.

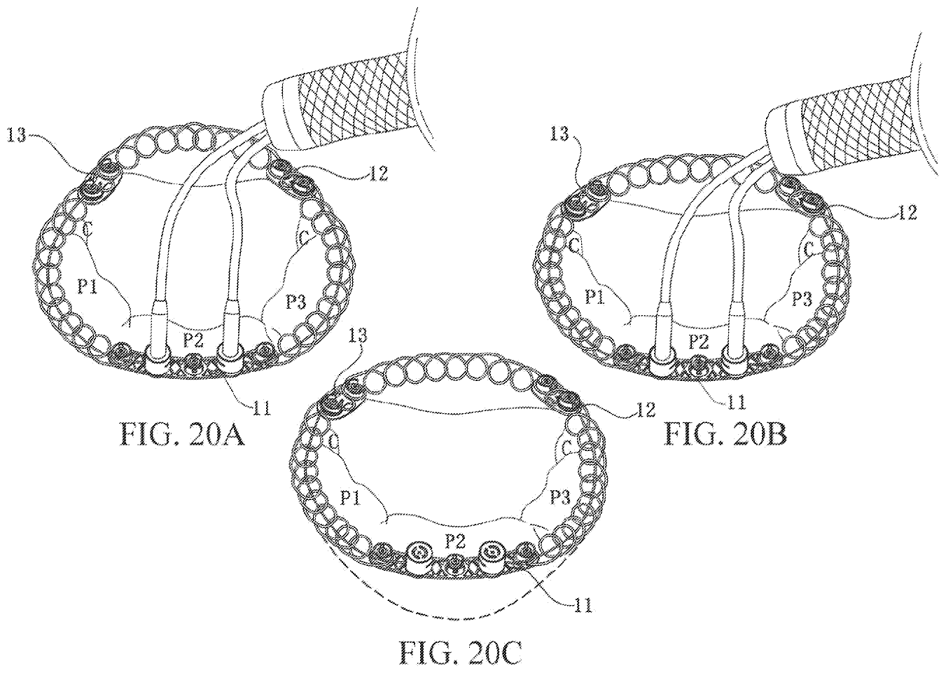

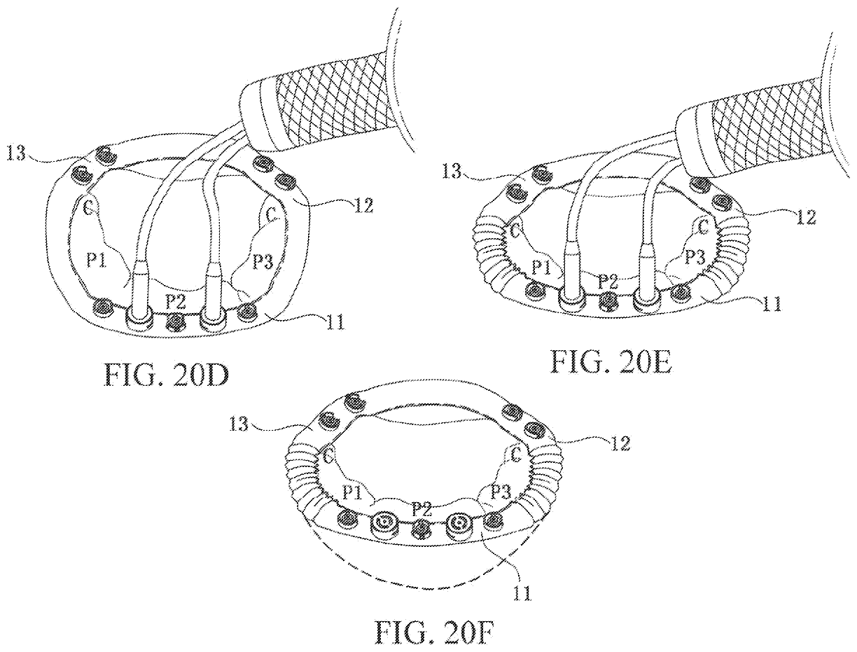

FIGS. 20A to 20F illustrate the change that occurs when the mitral valve annulus is adjusted using an annuloplasty device having, for example, three bar members as shown in FIG. 1A or FIG. 1B, wherein FIGS. 20A to 20C show changes in the extendable and compressible element, the interconnecting element, and the annulus tissue without being covered by the fabrics, FIGS. 20D to 20F show changes in the shaping ring and annulus tissue covered with the fabrics, wherein FIGS. 20A and 20D show the annuloplasty device before adjustment, FIGS. 20B and 20E show the annuloplasty device after adjustment, and FIGS. 20C and 20F show a comparison of the annuloplasty device before and after adjustment. It can be seen that the extendable and compressible element between the medial and lateral trigone regions and the P2 region of the mitral valve after adjustment becomes shorter, the interconnecting element between the medial and lateral trigone regions is almost unchanged, the distance between the annulus in the direction of the AP axis is linearly shorter, and anterior and posterior leaflets close together without folds.

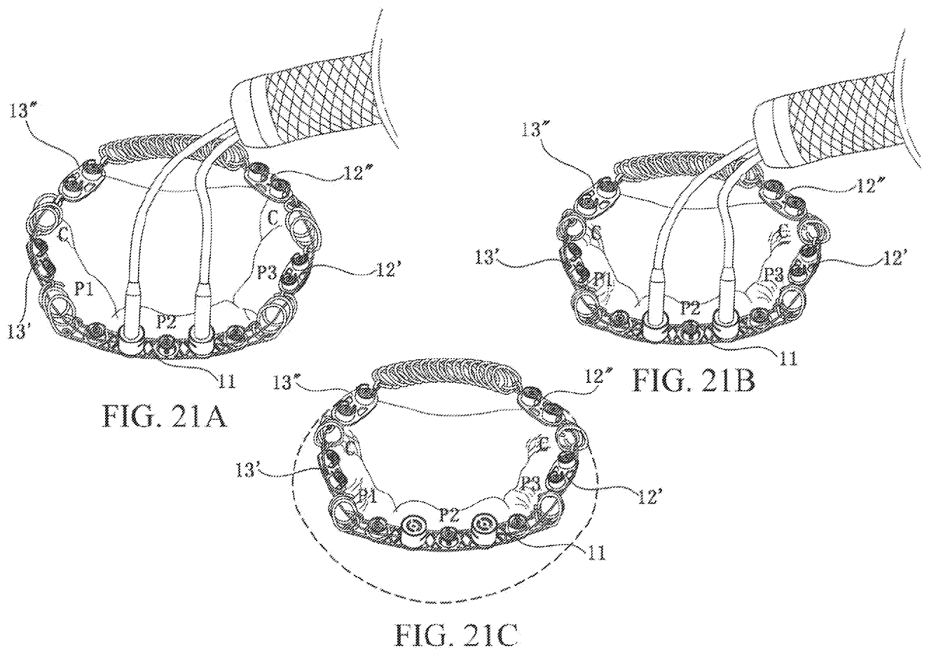

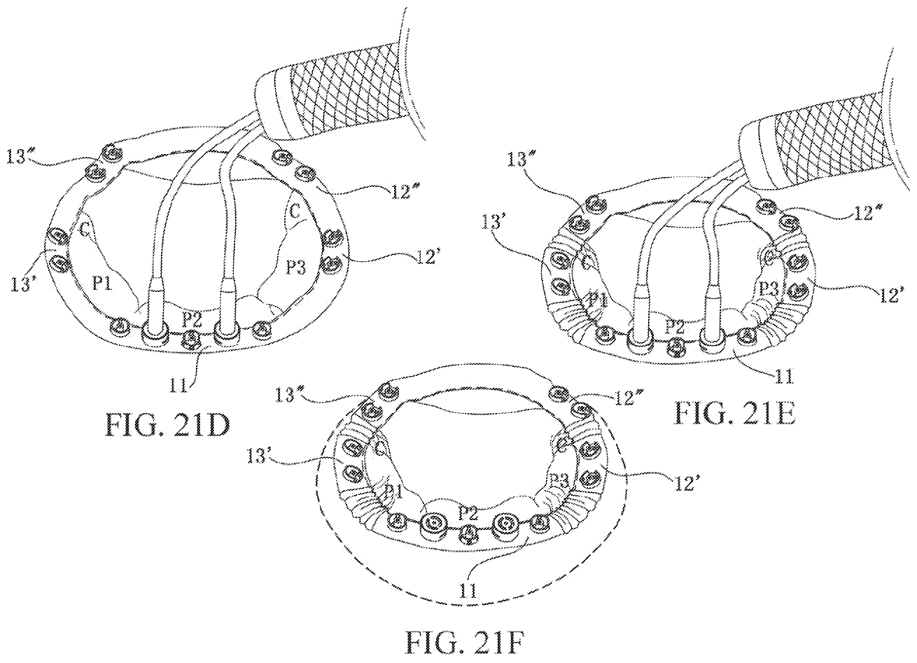

FIGS. 21A to 21F illustrate the change that occurs when the mitral valve annulus is adjusted using an annuloplasty device having, for example, five bar members as shown in FIG. 15A, wherein FIGS. 21A to 21C show changes in the extendable and compressible element, the interconnecting element, and annulus tissue without being covered by the fabrics, FIGS. 21D to 21F show changes in the shaping ring and annulus tissue covered with the fabrics, wherein FIGS. 21A and 21D show the annuloplasty device before adjustment, FIGS. 21B and 21E show the annuloplasty device after adjustment, and FIGS. 21C and 21F show a comparison of the annuloplasty device before and after adjustment. It can be seen that the extendable and compressible element between the medial and lateral trigone regions and the P1/P3 region of the mitral valve after adjustment becomes shorter. The extendable and compressible element between the P1/P3 region and the P2 region becomes shorter. There is little change in the interconnecting element between the medial and lateral trigone regions. The distance between the annulus in the direction of the AP axis becomes linearly shorter. The anterior and posterior commissure regions are annularly reduced, with the anterior and posterior leaflets closing together. The leaflets of P1 and P3 regions are creased, while the P2 region and the anterior leaflet are not creased.

An exemplary procedure for correcting the mitral valve using the shaping ring 1 will now be described with reference to FIG. 18.

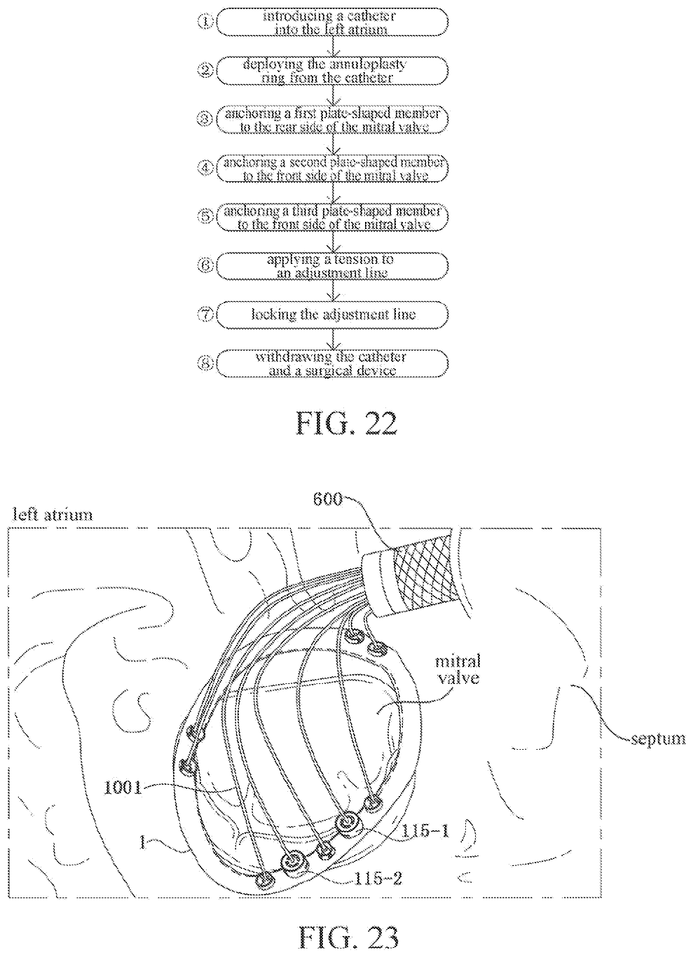

FIG. 22 shows an exemplary method of implanting the shaping ring 1 shown in one of FIGS. 1A, 1B, and 14A into a heart.

The step (1) of the method is to introduce the distal end of a delivery catheter 600 into the left atrium of the subject. This may be accomplished using a transseptal approach, a left atrial approach, or other approach to enter the left atrium. Hereinafter a specific procedure is described with the example of a transseptal approach in which the distal end of the catheter passes through the septa of the heart and into the left atrium of the subject. In some embodiments, an inner dilator (not shown) may be disposed in the distal end of the catheter for passage through the septa.

Once the distal end of the catheter is introduced into the left atrium, the shaping ring 1 is deployed from the distal end of the catheter in the step (2). In some embodiments, the shaping ring may be preloaded into a catheter (as shown in FIG. 17A) and advanced through the catheter into the left atrium. As shown in FIG. 17B, a guide 1001 may be removably attached to each of the guide engagement portions to push the shaping ring 1 through the catheter and deploy the shaping ring 1 from the distal end. In addition, the surgical apparatuses may be introduced into the body through a catheter. The surgical apparatuses may include a steerable sheath 700 (internally including a torque drive device 200) for adjusting the position and orientation of the bar member, a torque drive device 300 for driving the cinching device, and a drive tube 500 for transmitting torque to the anchoring element. The surgical apparatuses may also include other instruments required for annuloplasty.

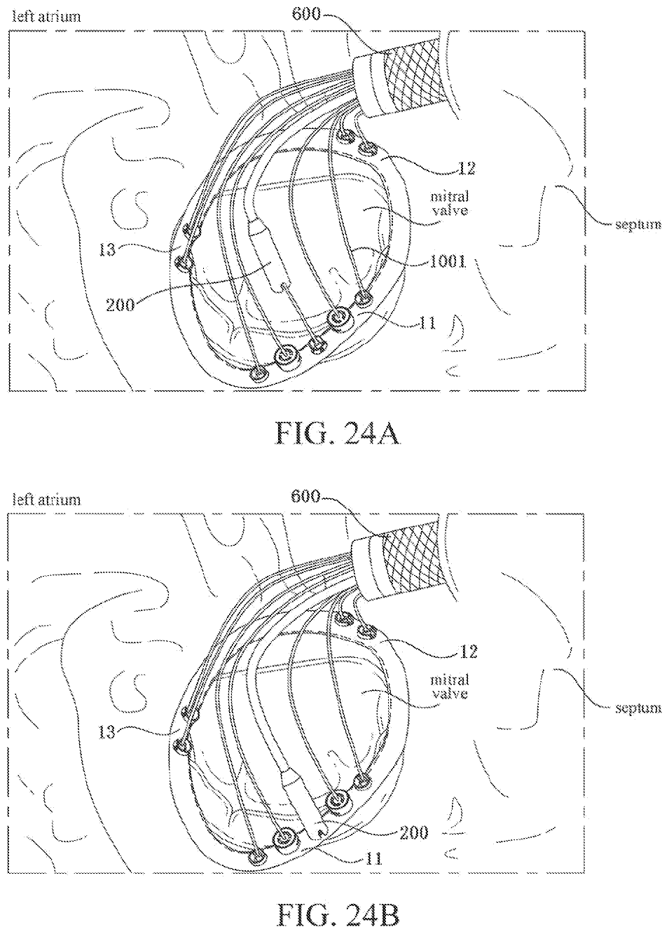

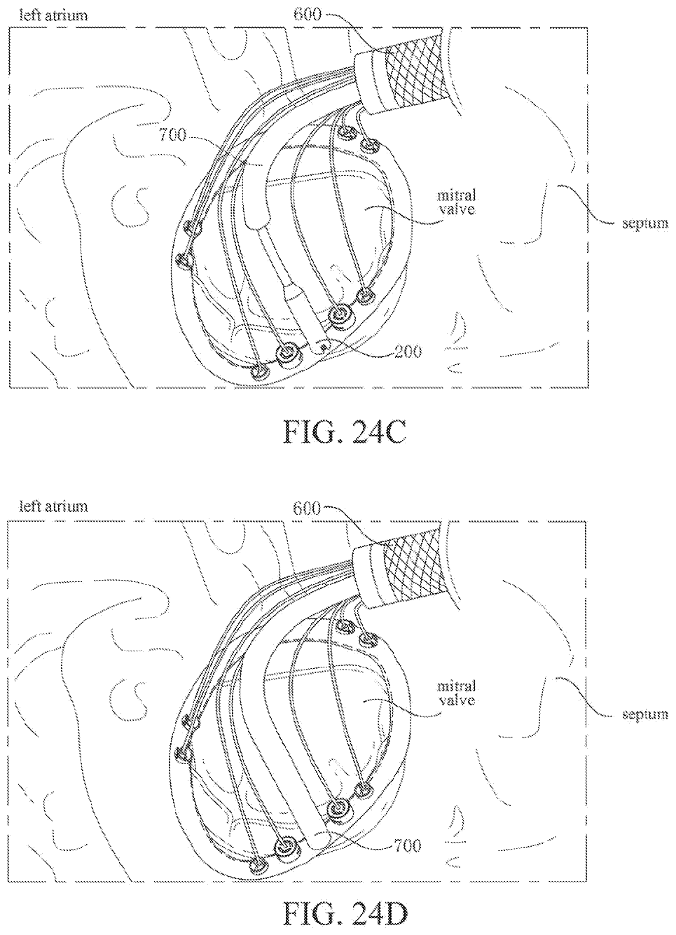

Referring to FIGS. 17C and 23, once the shaping ring 1 is fully extended from the distal end of the catheter 600, the folded shape can self-unfold into an original ring shape. As shown in FIGS. 24A to 24E, the torque drive device 200 may slide distally over the guide 1001 until the open slot 201 of the torque drive device 200 engages with the projections 1117/1217 of the anchor alignment member. The torque drive device 200 and the steerable sheath 700 may then be used in cooperation to position and rotate the individual bar members (e.g., the bar members 11, 12, 13) until the shaping ring 1 is adjusted to the desired implantation position and orientation.

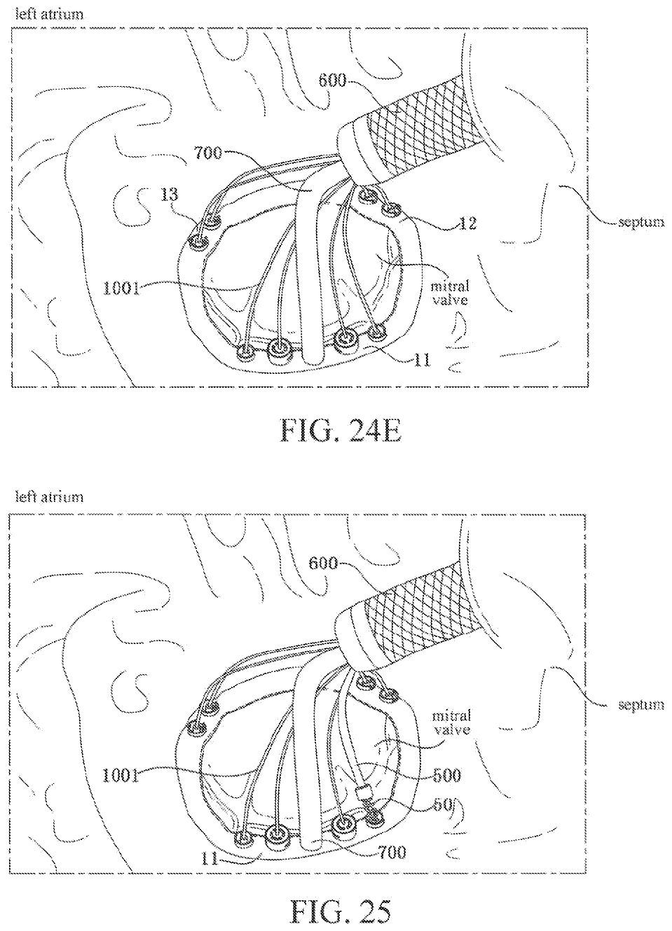

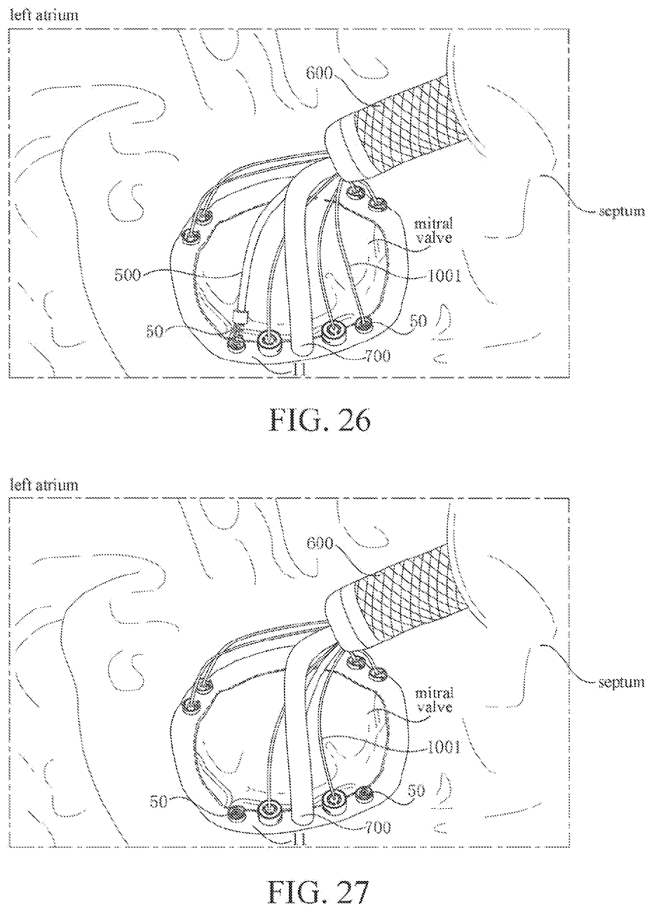

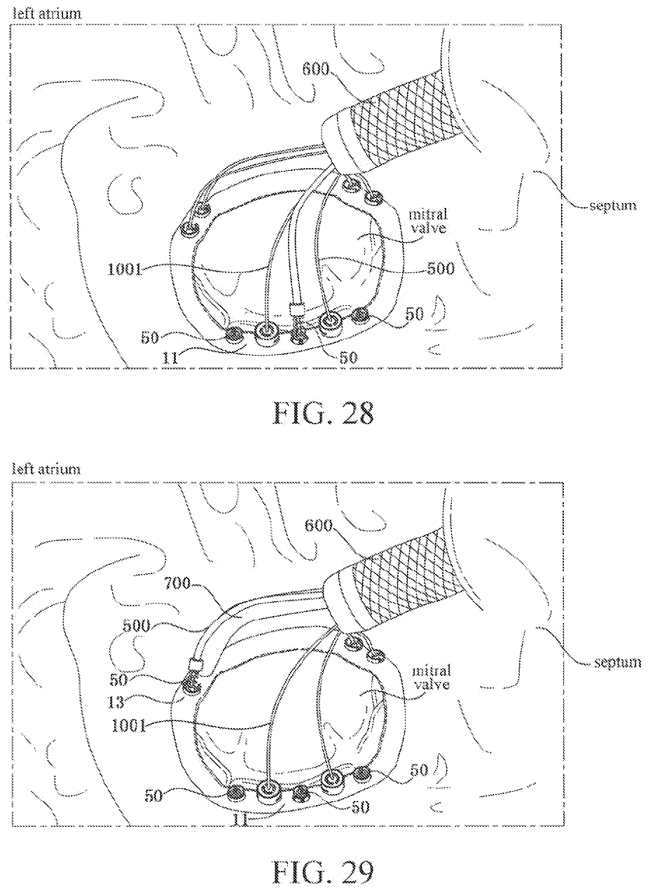

In the step (3), the bar member 11 is anchored to the posterior side of the mitral valve. In the step (4), the bar member 13 is anchored to the lateral trigone region of the mitral valve. In the step (5), the bar member 12 is anchored to the medial trigone region of the mitral valve. The specific anchoring method can be realized by the following means: firstly the drive tube 500 (for driving the tissue anchoring element to screw the tissue anchoring element into the tissue) is caused to slide on the guide attached to the anchoring position (the anchoring position is defined by the connection portion of the respective bar member, the same hereinafter) near the medial side end of the bar member 11. As shown in FIG. 25, the drive tube 500 has a helical tissue anchoring element (e.g., the helical tissue anchoring element 50) on its distal end. When the steerable sheath 700 is located in the middle of the bar member 11 and the bar member 11 is held against the mitral valve annulus tissue, the drive tube 500 may be rotated to screw the medial side tissue anchoring element through the bar member 11 into the underlying tissue (as shown in FIG. 26). The drive tube 500 may then be removed from the medial side tissue anchoring element and is caused (or another drive tube with another helical tissue anchoring element is caused) to slide on the guide attached to the anchoring position located near the lateral end of the bar member 11 (as shown in FIG. 26). When the lateral anchoring element and the steerable sheath 700 hold the bar member against the mitral valve annulus tissue, the drive tube 500 may be rotated to screw the outer tissue anchoring element through the bar member 11 into the underlying tissue (as shown in FIG. 27). The drive tube may then be removed from the outer anchoring element and is caused (or another drive tube with another helical tissue anchoring element is caused) to slide on the guide attached to the middle anchoring position (as shown in FIG. 28). In some embodiments, the steerable sheath may be held at the position against the bar member 11 when the central anchoring element is placed (as shown in FIG. 27). Alternatively, the steerable sheath may be removed from the bar member 11 before the drive tube and the middle anchoring element slide into engagement over the guide at the middle anchoring position (as shown in FIG. 28). When the lateral anchoring element and the medial side anchoring element hold the bar member 11 against the mitral valve annulus tissue, the drive tube 500 may be rotated to screw the middle anchoring element through the bar member into the underlying tissue. FIGS. 27 and 28 show the bar member 11 with the guide 1001 removed from the guide-engaging element at the anchoring position, such as by being screwed out. FIG. 29 shows a bar member 11 in which three tissue anchoring elements have been placed and all the guides at their anchoring positions have been removed.

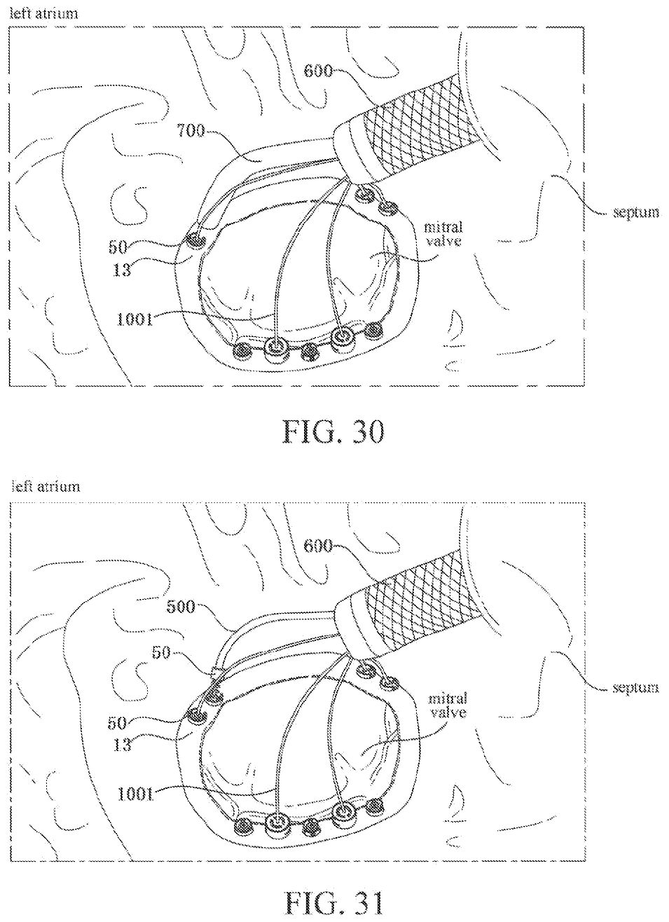

In the steps (4) and (5), when the bar member 13 is provided with two anchoring positions, before anchoring is performed at the previously mentioned two anchoring positions (as shown in FIG. 29), the steerable sheath 700 is first positioned in an anchoring position on the bar member 13, and when the bar member 13 is held against the mitral valve annulus tissue, the drive tube 500 may be rotated to screw the anchoring element into the underlying tissue through the other anchoring position of the bar member 13. As shown in FIG. 30, the drive tube 500 may then be removed from the anchored anchoring element and is caused (or another drive tube with another helical tissue anchoring element is caused) to slide on the guide attached to the unanchored anchoring position of the bar member 13 (as shown in FIG. 31). In some embodiments, the steerable sheath 700 may be held at the position against the bar member 13 when anchoring element is placed (as shown in FIG. 30) at one side. Alternatively, the steerable sheath 700 may be removed from the bar member 13 before the drive tube 500 and the anchoring element slide into engagement over the guide at the predetermined anchoring position (as shown in FIG. 31). After the anchoring element of one side holds the bar member 13 against the mitral valve annulus tissue, the drive tube 500 may be rotated to screw anchoring element of the other side through the bar member 13 into the underlying tissue.

When the bar member 13 is provided with an anchoring position, the steerable sheath 700 is positioned on the anchoring position of the bar member 13, and when the bar member 13 is held against the mitral valve annulus tissue, the drive tube 500 may be used within the steerable sheath 700 to screw the anchoring element through the bar member 13 into the underlying tissue.

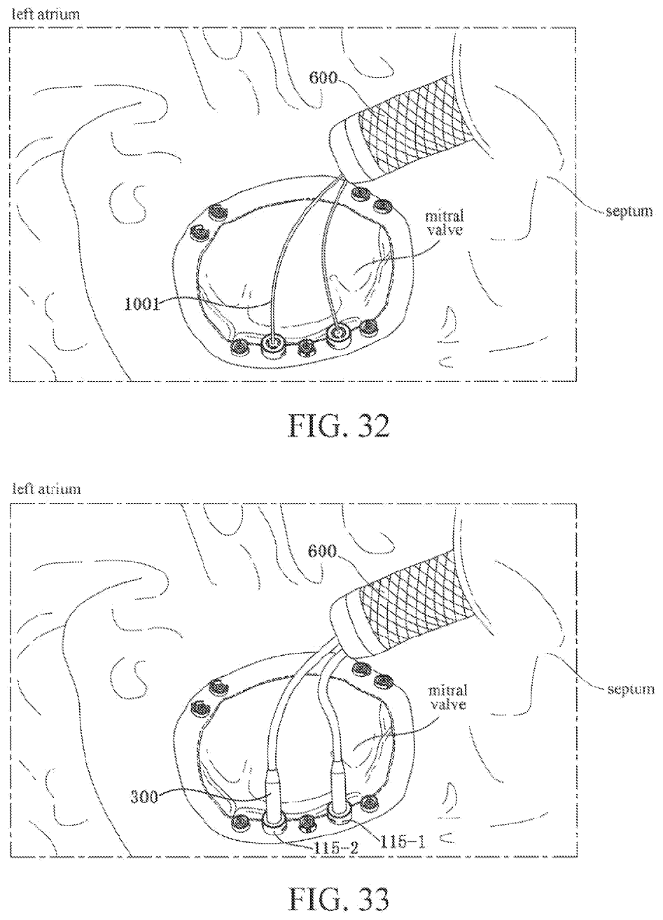

The anchoring process of the bar member 12 is similar to that of the bar member 13. FIG. 32 shows a schematic diagram in which the bar members 11, 12 and 13 are all anchored to the annulus tissue.

In an optional next step, the tissue anchoring elements are anchored in the P1 and P3 regions, and the shaping rings in the corresponding regions are anchored to the annulus tissue.

Once the shaping ring is anchored well, the operation of the step (6) may be performed to apply the additional tension to the adjustment wires 171, 172 to pull the anterior and posterior mitral valves together. In some embodiments, the tension in the adjustment wires 171, 172 may increase simultaneously. In some embodiments, the tension in the adjustment wire may increase incrementally, with the increase occurring alternately between the two wires until a desired size of the mitral valve annulus is achieved. In some embodiments, the final tension and/or the achieved tissue approach of each adjustment wire is approximately the same. In some embodiments, the final tensions and/or the achieved tissue approaches of the adjustment wires 171, 172 are different. This is generally possible for all of the systems disclosed herein. In some embodiments, real-time echocardiography of the mitral valve may be employed to monitor whether the reduction in mitral valve regurgitation is achieved as desired when the adjustment wires 171, 172 are tightened.

After the desired size of the annulus is obtained, the process goes into the operation of the step (7) and the adjustment wires 171, 172 may be knotted. In some embodiments, a reversible lock may be used during the cinching process, and the reversible lock is configured to permanently maintain the position of the adjustment wire. A disconnection member may be used to cut away the redundant portion of the adjustment wire to maintain the working adjustment wire remaining in the body.

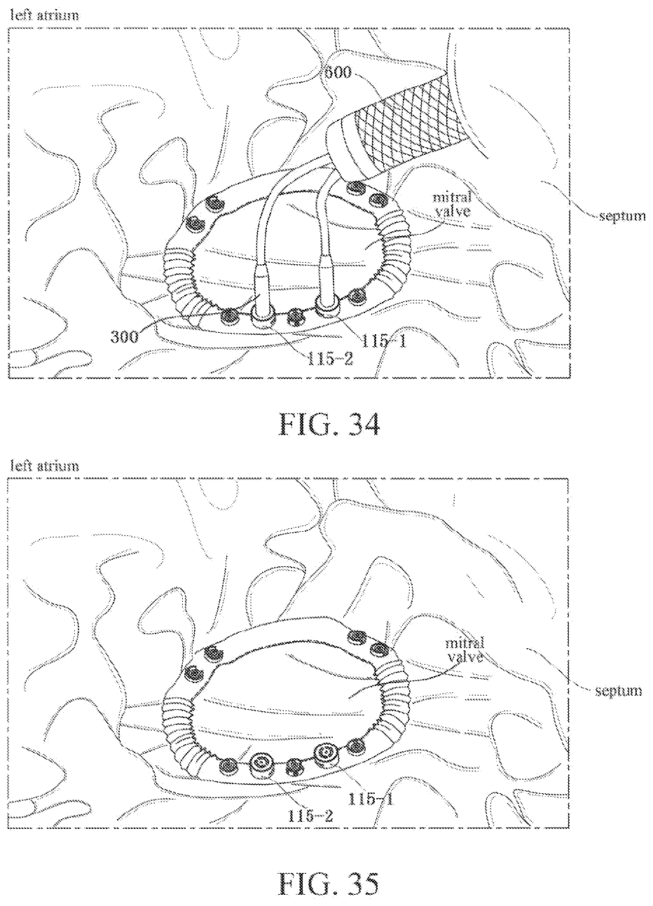

In the annuloplasty ring provided with the cinching device, as shown in FIGS. 13A to 13C, 33 and 34, the torque drive device 300 firstly slides on the guide attached to the cinching device. Then the torque drive device 300 is continued to be manipulated to move downward such that the elastic sheet is separated from the one-way ratchet teeth. The torque drive device 300 is manipulated to drive the reel to rotate clockwise or counterclockwise until proper tension and/or approach of the annulus tissue is achieved. After the removal of the torque drive device, as shown in FIG. 35, the ratchet teeth of the reel is locked by the elastic sheet, and the cinching device is in a locked state, thereby maintaining the tension of the adjustment wire.

The catheter, along with the steerable sheath, can then be withdrawn from the left atrium (in the step (8) shown in FIG. 22).

In addition to tensioning the shaping ring 1 during the surgical procedure, the previous cinching device can also be re-tensioned at a later stage to further adjust the size of the annulus.

It should be noted that the torque drive device 200 for adjusting the position and direction of the bar member and the torque drive device 300 for driving the cinching device may be the same, that is, the open slot 301 may replace the open slot 201.

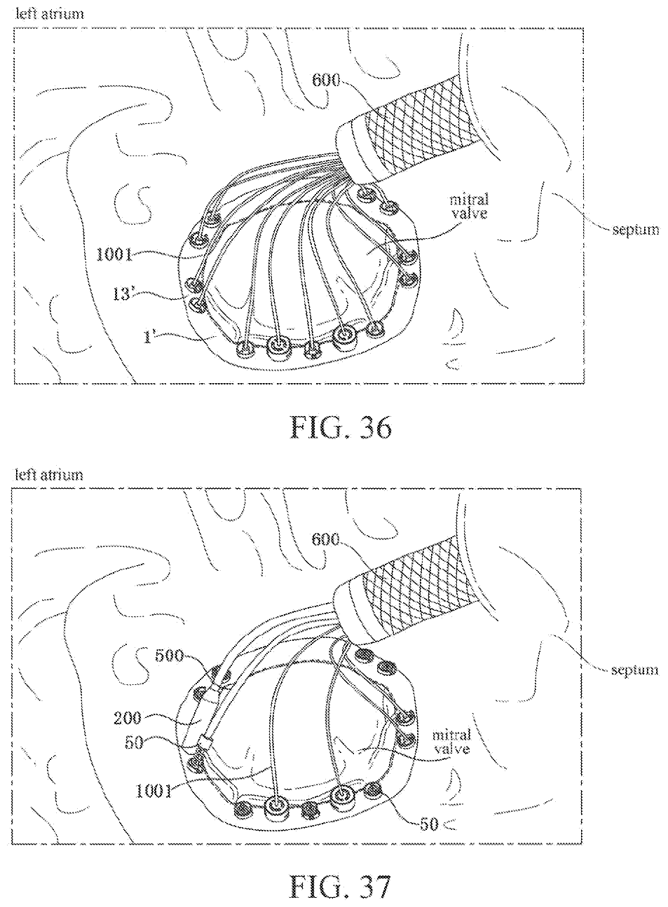

For the implantation of the above-described shaping ring 1′, after anchoring the bar member 11 and the two bar members 12″, 13″, the two newly added bar members 12′, 13′ are anchored, wherein the bar member 12′ is anchored in the region P3 and the bar member 13′ is anchored in the region P1, as shown in FIGS. 36 and 37.

After the bar members are all fixed to the mitral valve annulus tissue, the adjustment wires 171, 172 are respectively tensioned to pull the bar members 12′, 12″, 13′, 13″ and the bar member 11 toward each other, and the anterior and posterior leaflets of the mitral annulus are closer together, and the annulus is contracted, thereby reducing and eliminating regurgitation. Finally, the adjustment wires 171, 172 are locked to maintain the tightened position.

The process of implantation of the above described shaping ring 1″ is similar to that of the shaping ring 1′ except that the four adjustment wires 17-1′, 17-2′, 17-2′, 17-2″ need to be tightened.

The specific embodiment of the shaping ring according to embodiment of the present disclosure has been described above with reference to the accompanying drawings. However, these descriptions are merely for purposes of describing the basic principles of the disclosure and applications thereof, and are not intended to limit the scope of the disclosure. The scope of the present disclosure is defined merely by the appended claims and their equivalents. Many different embodiments may be envisaged by those skilled in the art in view of the present disclosure.

For example, those skilled in the art may also easily provide different numbers of bar members and/or provide different numbers of cinching devices on different bar members and/or provide different adjustment wires to adjust the length of a corresponding number of extendable and compressible elements according to the teaching of the present disclosure. Such modifications or variations are within the scope of the present disclosure.

1. An annuloplasty device configured to be transcatheter implanted, comprising:
a shaping ring including an extendable and compressible element, a first bar member and a second bar member each connected to the extendable and compressible element, the first bar member and the second bar member each provided with at least one connection portion, and a first adjustment wire configured to pull the first bar member and the second bar member toward each other when tightened;
a first tissue anchoring element configured to fix the first bar member to an annulus tissue via the connection portion of the first bar member; and
a second tissue anchoring element configured to fix the second bar member to the annulus tissue via the connection portion of the second bar member.

2. The annuloplasty device according to claim 1, wherein the shaping ring further includes a third bar member that is connected to the first bar member and the second bar member by the extendable and compressible element;
wherein the shaping ring further includes a second adjustment wire configured to pull the first bar member and the third bar member toward each other when tightened.

3. The annuloplasty device according to claim 2, wherein the first bar member is configured to be anchored to the annulus tissue at least two ends by a tissue anchoring element via respective connection portions.

4. The annuloplasty device according to claim 3, wherein the shaping ring further includes a fourth bar member and a fifth bar member, wherein the fourth bar member is connected to the extendable and compressible element between the first bar member and the second bar member, the fifth bar member is connected to the extendable and compressible element between the first bar member and the third bar member.

5. The annuloplasty device according to claim 3, wherein a tissue anchoring element is provided at a position between the first bar member and the second bar member where the extendable and compressible element is located, and a tissue anchoring element is provided at a position between the first bar member and the third bar member where the extendable and compressible element is located, to anchor the extendable and compressible element to the annulus tissue.

6. The annuloplasty device according to claim 1, wherein at least one of the first bar member and the second bar member is provided with a cinching device configured to tighten the first adjustment wire, thereby adjusting the size of the annulus.

7. The annuloplasty device according to claim 6, wherein the cinching device includes: a housing fixed to the first or second bar member provided with the cinching device; a reel provided with ratchet teeth, the reel being accommodated in the housing in such a manner as to be rotatable with respect to the housing; and an elastic sheet having a ratchet engaging side and a fixing side fixed to the housing.

8. The annuloplasty device according to claim 7, wherein the ratchet teeth are one-way ratchet teeth, and the reel is one-way locked when the ratchet engaging side of the elastic sheet is engaged with the ratchet teeth.

9. The annuloplasty device according to claim 1, wherein the extendable and compressible element includes an inclined coil element configured to be inclined with respect to a longitudinal centerline of the extendable and compressible element so that the extendable and compressible element can snugly abut against a surface of the annulus tissue.

10. The annuloplasty device according to claim 9, wherein the first adjustment wire is at least partially received within a cavity defined by the coil element.

11. The annuloplasty device according to claim 2, wherein the shaping ring further includes an interconnecting element configured to interconnect the second bar member and the third bar member such that the shaping ring forms a complete annular shape, wherein the shape of the interconnecting element is substantially constant before and after adjustment of the shaping ring.

12. The annuloplasty device according to claim 11, wherein the interconnecting element includes an inclined coil element configured to be inclined with respect to a longitudinal centerline of the interconnecting element so that the interconnecting element can snugly abut against a surface of the annulus tissue.

13. The annuloplasty device according to claim 11, wherein the interconnecting element is configured to have a shape corresponding to a portion of the physiological annulus against which it abuts snugly.

14. The annuloplasty device according to claim 2, wherein the first bar member includes at least two sub bar members which are hinged to each other such that the at least two sub bar members can be folded on each other.

15. An annuloplasty device configured to be transcatheter implanted, comprising:
a shaping ring including an extendable and compressible element and least one bar member connected to the extendable and compressible element, the at least one bar member is provided with at least one connection portion; and
a tissue anchoring element configured to fix the at least one bar member to an annulus tissue via the connection portion,
wherein the connection portion includes an abutment fixed to a hole wall of a through hole of the bar member and a cylindrical anchor alignment member aligned with the through hole, the cylindrical anchor alignment member is configured to cause the tissue anchoring element to be substantially perpendicular to a bottom surface of the at least one bar member in contact with the annulus tissue when the tissue anchoring element is anchored.

16. The annuloplasty device according to claim 15, wherein the at least one bar member includes a position adjustment structure configured to adjust the position and/or orientation of the bar member.

17. The annuloplasty device according to claim 16, wherein the position adjustment structure includes at least one projection disposed on an outer surface of the cylinder wall of the cylindrical anchor alignment member.

18. The annuloplasty device according to claim 15, wherein the abutment is configured as a cross bar, the connection portion further includes a guide-engaging element disposed on the cross bar and the guide-engaging element is configured to rotate about an axis of the cross bar, the guide-engaging element is provided with a structure configured to be detachably connected with a guide for delivering the shaping ring.

19. The annuloplasty device according to claim 18, wherein at least one notch configured to receive the guide-engaging element when the guide-engaging element is tilted is provided in the cylinder wall of the cylindrical anchor alignment member.

20. The annuloplasty device according to claim 19, wherein the cross bar is provided with a limiting portion configured to align the guide-engaging element with the at least one notch.

21. A method of implementing annuloplasty, the method comprising:
connecting a guide to a guide-engaging element of a shaping ring;
folding the entire shaping ring into a catheter;
introducing the shaping ring and surgical apparatuses through the catheter into a body of a patient;
anchoring a bar member of the shaping ring to annulus tissue by a tissue anchoring element;
contracting the annulus to a proper size by adjusting an adjustment wire;
locking the adjustment wire to keep the annulus properly sized; and
withdrawing the catheter and the surgical apparatuses.

22. The method according to claim 21, wherein the method further comprises: adjusting the angle and/or orientation of the bar member by engaging a torque drive device with a position adjustment structure on the bar member before anchoring the shaping ring to the annulus tissue.

23. The method according to claim 22, wherein the bar member includes a first bar member, a second bar member and a third bar member, the method is used to repair a mitral valve and further comprises:
fixing the first bar member to or near region P2 of a posterior leaflet of the annulus tissue by a tissue anchoring element;
anchoring the second bar member to a vicinity of a medialtrigone region of the annulus tissue by a tissue anchoring element;
anchoring the third bar member to a vicinity of a lateral trigone region of the annulus tissue by a tissue anchoring element; and
adjusting an adjustment wire by a cinching device rotatably provided on at least one of the first bar member, the second bar member, and the third bar member.

24. The method according to claim 23, wherein the bar member further includes a fourth bar member and a fifth bar member, the method further comprising:
anchoring the fourth bar member to or near region P3 of the posterior leaflet of the annulus tissue by a tissue anchoring element;
anchoring the fifth bar member to or near region P1 of the posterior leaflet of the annulus tissue by a tissue anchoring element; and
adjusting the adjustment wire by rotating the cinching device to reduce the size of the physiological annulus.

25. The method according to claim 23, the method further comprising:
anchoring the shaping ring between the first bar member and the second bar member to the annulus tissue by a tissue anchoring element;
anchoring the shaping ring between the first bar member and the third bar member to the annulus tissue by a tissue anchoring element; and
adjusting the adjustment wire by rotating the cinching device to reduce the size of the physiological annulus.

26. The method according to claim 21, the method further comprising a step of assembling the bar member, an extendable and compressible element, the adjustment wire and a cinching device of the shaping ring before loading the shaping ring into the catheter.

27. The annuloplasty device according to claim 11, wherein the shaping ring does not include an adjustment wire configured to pull the second bar member and the third bar member toward each other.

28. An implantable annuloplasty system comprising:
a first member;
at least one first member anchor configured to anchor the first member to a posterior side of a mitral valve annulus in a left atrium of a heart;
a second member that is separate from the first member and is configured to not directly contact the first member;
at least one second member anchor configured to anchor the second member to an anterior side of the mitral valve annulus;
a third member that is separate from the first member and the second member and is configured to not directly contact the first member or the second member;
at least one third member anchor configured to anchor the third member to the anterior side of the mitral valve annulus;
a first extendable and compressible element spanning between the first member and the second member;
a second extendable and compressible element spanning between the first member and the third member;
a third extendable and compressible element spanning between the second member and the third member;
a first flexible tensile member configured to span directly between the first member and the second member at least partially through the first extendable and compressible element such that tension may be applied to the first tensile member to draw the first member and the second member toward one another and bring the posterior side and the anterior side of the mitral valve annulus into closer approximation; and
a second flexible tensile member configured to span directly between the first member and the third member at least partially through the second extendable and compressible element such that tension may be applied to the second tensile member independently from the tension applied to the first tensile member to draw the first member and the third member toward one another and bring the posterior side and the anterior side of the mitral valve annulus into closer approximation,
wherein all elements of the implantable annuloplasty system are configured to be deployed into the left atrium through a catheter.

29. The implantable annuloplasty system according to claim 28, wherein there is no tensile member configured to span directly between the second member and the third member such that tension may be applied to the tensile member to draw the second member and the third member toward one another.

30. The implantable annuloplasty system according to claim 28, wherein at least one of the first, the second and the third extendable and compressible elements includes an inclined coil element configured to be inclined with respect to a longitudinal centerline of the extendable and compressible element so that the extendable and compressible element can snugly abut against a surface of annulus tissue.

31. The implantable annuloplasty system according to claim 28, wherein each of the first, the second and the third extendable and compressible elements includes an inclined coil element configured to be inclined with respect to a longitudinal centerline of the extendable and compressible element so that the extendable and compressible element can snugly abut against a surface of annulus tissue.

32. The implantable annuloplasty system according to claim 28, wherein the first flexible tensile member does not directly contact the second flexible tensile member.
